SEQUENCE LISTING (9 sequences)

SEQ ID NO: 1 - DNA, 439 bp, Homo sapiens
  CDS (11)..(328)
agcagccaagatggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaatcatgttttctgaaaatataaccagccattggctatttaaaacttgtaatttttttaatttacaaaaatataaaatatgaagacataaacccagttgccatctgcgtgaca

SEQ ID NO: 2 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 3 - DNA, 453 bp, Escherichia coli
  CDS (118)..(447)
tcagcttaactattgctttacgaaagcgtatccggtgaaataaagtcaacctttagttggttaatgttacaccaacaacgaaaccaacacgccaggcttattcctgtggagttatatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgtaagggaat

SEQ ID NO: 4 - protein, 109 aa, Escherichia coli
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 5 - DNA, 506 bp, Macaca mulatta
  CDS (64)..(381)
gaattcgctttggatccatttccatcggtccttacagccgctcctcagaccccagcagcaaagatggtgaagcagatcgagagcaaggctgcttttcaggaagccttggacgacgcaggtgataaacttgtagtagttgacttctcagccacgtggtgtgggccttgcaaaatgatcaagcctttctttcattccctctctgaaaagtattccaacgtggtattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaatcatgttttctgaaaacataaccagccattggctatttaaaacttgtaatttttttaatttacaaaaatataaaatatgaagacataaccagttgccatctgcgtgacaataaacattatgctaa

SEQ ID NO: 6 - protein, 105 aa, Macaca mulatta
MVKQIESKAAFQEALDDAGDKLVVVDFSATWCGPCKMIKPFFHSLSEKYSNVVFLEVDVDDCQDVASECEVKCMPTFQFFKKGQKVGEFSGANKEKLEATINELV

SEQ ID NO: 7 - DNA, 1281 bp, Rattus norvegicus
  CDS (56)..(556)
cccttgacgggcaggccggctgggctctgcatccctctgctcacactgccgggagatggctcagcggcttctcctgaggaggttcctgacctcagtcatctccaggaagcctcctcagggtgtgtgggcttccctcacctctacgagcctgcagacccctccgtacaatgctggtggtctaactggaacacccagccctgcccggacatttcacaccaccagagtctgttcaacaacctttaacgtccaggatggacctgactttcaagacagagttgtcaacagtgagacaccagttgtcgtggactttcatgcacagtggtgtggcccctgcaagatcctaggacctcggttagagaagatggtagccaaacagcacgggaaggtggtgatggccaaagtggacattgacgatcacacagaccttgccattgagtacgaggtgtctgctgtgcctaccgtgctggccatcaagaacggggacgtggtggacaagtttgtggggatcaaggacgaagaccagctggaagccttcctgaagaagctaattggctgacaagcagggaagaggccagactgcccttgcctgctggaaacccatttggggaggaacctagtagaactcccagccctcagctgtcatccttcgtgccctgcccctgttttgtccttgtgggtcttgcctttgggcagcagacttccaaacctagaagccaccagcacttcagagccagccctcagccaggatggccaggagaggggcaatgctgccgatggtgtggaggaggggcatcctgagcacctcctactgtgtgcttctcctgtgcacctactgtgtgcttcttctagggcttgttgctattcccctagatgctagacaaagcctgggccagcctgtattcaccgcccaggcaatggcccagtcctcgtctctgaggtcctctgatctgaagcctcagccccacacacacacatgtggctctggtgtatttcctctcctgcctctgcttgaaaaggaaacaagaagctgaacaagtgagagtaatggataattctctcatcttttgtaggtctgtaataaagaactttatgtgatcccttctacctcttgctgtgaagaacagagcccaccccattccctatctctgtcccttctcttccttgtcacctccctgggttagctgggccttggttctggttccctgagcaggaggaggagctgtgaacaagcagaccaataaaaccaggtttgcattgccccaaaaaaaaaaaaaaaaaaaa

SEQ ID NO: 8 - protein, 166 aa, Rattus norvegicus
MAQRLLLRRFLTSVISRKPPQGVWASLTSTSLQTPPYNAGGLTGTPSPARTFHTTRVCSTTFNVQDGPDFQDRVVNSETPVVVDFHAQWCGPCKILGPRLEKMVAKQHGKVVMAKVDIDDHTDLAIEYEVSAVPTVLAIKNGDVVDKFVGIKDEDQLEAFLKKLIG

SEQ ID NO: 9 - DNA, 19 bp, Artificial Sequence
  Description of Artificial Sequence  Synthetic Primer
ggcatgtccgggcatgtcc

(19) United States
(12) Patent Application Publication
(10) Pub. No.: US 2005/0208037 A1
(43) Pub. Date: Sep. 22, 2005

(54) Thioredoxin increases redox-cycling of anticancer agents thereby sensitizes cancer cells to apoptosis

(75) Inventors: Ravi Dashnamoorthy, Little Rock, AR (US); Kumuda C. Das, Little Rock, AR (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 10/970,370

(22) Filed: Oct. 21, 2004

Related U.S. Application Data
(60) Provisional application No. 60/513,134, filed on Oct. 21, 2003.

(51) Int. Cl. A61K 38/44; A61K 31/704

(52) U.S. Cl. 424/94.4; 514/34

(57) ABSTRACT

The present invention provides for treatment of cancer by enhancing the effectiveness of anticancer agents. The present invention therefore provides methods of increasing the apoptotic potential of anticancer drugs by increasing the expression of the cellular redox protein thioredoxin or thioredoxin-like molecules and thereby sensitizing the cancer to the anticancer agent. The present invention also provides methods of ameliorating negative side-effects of an anticancer therapy comprising a thioredoxin or a thioredoxin-like molecule and an anticancer therapy.

This application claims the benefit of U.S. Provisional Application Ser. No. 60/513,134 filed Oct. 21, 2003, the entire disclosure of which is specifically incorporated herein by reference.

The government owns rights in the present invention pursuant to grant number RPG-00-335-01 from the American Cancer Society.

### BACKGROUND OF THE INVENTION

1. Field of the Invention

The present invention relates generally to the fields of cancer biology and cancer therapy. More particularly, the present invention concerns compositions and methods for the treatment of neoplastic diseases.

2. Description of Related Art

Programmed cell death is a major mechanism of cancer cell destruction by chemotherapeutic and radiotherapeutic agents. However, to more effectively treat and eradicate this disease, agents that increase the apoptotic potential of cancer cells to anticancer agents are needed.

A growing body of evidence indicates that cellular reduction/oxidation (redox) status regulates various biologic processes, including signal transduction, gene expression, and cell proliferation. Oxidative stress can elicit positive cellular responses such as cellular proliferation or activation, as well as negative cellular responses such as growth inhibition or cell death. Thus, agents that enhance the redox-cycling of anticancer agents may prove therapeutically beneficial due to an ability to enhance the effectiveness of anticancer agents and promote cancer cell killing.

Thioredoxin (Trx) was originally identified in Escherichia coli (Holmgren et al., 1985). E. coli thioredoxin is a homodimer comprising of 35 kDa subunits with two redox-active cysteine residues at position 32 and 35 (Powis et al., 1995). Human thioredoxin was originally discovered as a T cell growth factor secreted from HTLV-I-infected T cell (Tagaya et al., 1989). The human thioredoxin gene has been provisionally mapped to 3p11-p12. Thioredoxin, a small protein of approximately 100 amino acid residues, is ubiquitously present and is evolutionarily conserved from prokaryotes to higher eukaryotes, plants, and animals (Holmgren, 1984; Holmgren, 1985; Holmgren, 1989). Thioredoxin is a 12 kDa ubiquitous protein that controls the redox state of various target proteins by means of thiol-disulfide exchanges as a constituent of cellular antioxidant defense systems (Holmgren, 1985; Holmgren, 1995). The redox-active sulfhydryls in thioredoxin are located at a highly-conserved active-site sequence -Trp-Cys-Gly-Pro-Cys- (Holmgren, 1995). The pathway for the reduction of a protein disulfide by thioredoxin entails nucleophilic attack by one of the active-site sulfhydryls, formation of a protein-protein disulfide, and subsequent intramolecular displacement of the reduced target proteins with concomitant formation of oxidized thioredoxin (Holmgren, 1995; Fernando et al., 1992).

Thioredoxin exists in either a reduced form or an oxidized form. Oxidized thioredoxin (Trx-S₂), where the two cysteine residues are linked by an intramolecular disulfide bond, is reduced by flavoenzyme thioredoxin reductase (TR) and NADPH (Holmgren, 1985). Reduced thioredoxin (Trx-(SH)₂) contains two thiol groups and can efficiently catalyze the reduction of many exposed disulfides. Thus, thioredoxin participates in various redox reactions via the reversible oxidation and reduction of the two cysteine residues in the active center.

Besides being an antioxidant itself (Das and Das, 2000; Mitsui et al., 1992), thioredoxin also plays an important role in regulating the expression of other antioxidant genes, such as manganese superoxide dismutase (Das et al., 1997). It has been reported that thioredoxin scavenges hydroxyl radicals and quenches singlet oxygen using EPR spectrometry (Das and Das, 2000). However, thioredoxin was found not to scavenge superoxide anions (Das and Das, 2000).

In eukaryotic cells, thioredoxin has been implicated in a wide variety of biochemical and biological functions. It can function as a hydrogen donor, similar to the prokaryotic thioredoxin (Holmgren, 1985). In addition, thioredoxin can facilitate refolding of disulfide-containing proteins (Lundstrom and Holmgren, 1990) and modulate the activity of some transcription factors such as NFκB and AP-1 (Meyer et al., 1993; Schenk et al., 1994). Thioredoxin is an efficient antioxidant, which can reduce hydrogen peroxide (Spector et al., 1988), scavenge free radicals (Schallreuter and Wood, 1986), and protect cells against oxidative stress (Nakamura et al., 1994). Another role of thioredoxin is the growth stimulation of human T cells. Furthermore, thioredoxin reportedly inhibits the expression of human immunodeficiency virus in macrophages (Newman et al., 1994).

Redox activity is essential for growth stimulation by thioredoxin. Thioredoxin has been shown to stimulate the growth of various normal and cancer cell lines (Wakasugi et al., 1990; Yodoi and Tursz, 1991; Oblong et al., 1994; Gasdaska et al., 1995). Thioredoxin is secreted by tumor cells and stimulates cancer cell growth. Increased expression of thioredoxin has been observed in primary lung cancer, colon cancer, cervical neoplastic squamous cells and hepatocellular carcinoma (Berggren et al., 1996; Fujii et al., 1991; Nakamura et al., 1992). It has been reported that the transfection of dominant negative mutant thioredoxin reversed a transformed phenotype of ER-positive human breast cancer cell line, MCF-7, suggesting that endogenous thioredoxin plays an important role in malignant development of breast cancer (Gallegos et al., 1996). However, the endogenous molecular targets of thioredoxin in breast carcinogenesis have not yet been identified. A class of disulfide inhibitors of thioredoxin has been identified. These disulfides inhibit cancer cell growth in culture and have antitumor activity against some human tumor xenografts in animals (Powis et al., 1998).

It is known in the art that cellular redox status is maintained by intracellular redox-regulating molecules, such as thioredoxin. However, the full role of thioredoxin in cells is undefined. Thioredoxin is a major redox-regulatory molecule which, via dithiol/disulfide exchange activity, determines the oxidation state of protein thiols. Thioredoxin is widely considered an antioxidant enzyme that protects the cells from various oxidative stresses (Holmgren and Bjornstedt, 1995; Powis and Montfort, 2001). A thioredoxin system comprised of thioredoxin and thioredoxin reductase plays a role in the regulation of cell proliferation and gene transcription (Powis and Montfort, 2001). Thioredoxin has been shown to confer resistance against ROS-generating anticancer drugs (Sasada et al., 1996; Yokomizo et al., 1995). Conversely, recent studies have also shown that thioredoxin does not confer resistance against doxorubicin (Berggren et al., 2001). Additionally, thioredoxin does not protect MCF-7 cells from apoptosis induced by ROS generating drugs (Berggren et al., 2001).

Anthracyclines are a class of antitumor agents that undergo redox-cycling in living cells, producing increased amounts of reactive oxygen species and semiquinone radical, both of which can cause DNA damage, and consequently trigger p53-mediated apoptotic death of cancer cells. These agents have been effectively used in the clinic as chemotherapeutic agents to treat a variety of cancers (Gluck, 2002; Weiss, 1992). However, their use has been limited due to acute and chronic cardiotoxicity that can occur at doses that are effective for cancer chemotherapy (Mordente et al., 2001; Hrdina et al., 2000; Childs et al., 2002).

In view of the above, methods and compositions for increasing the effectiveness of anticancer agents are needed, particularly agents that might be affected by redox-cycling.

### SUMMARY OF THE INVENTION

The present invention overcomes the deficiencies in the art by providing a novel means of enhancing the effectiveness and benefits of anticancer agents. Thus, the present invention provides a method of treating a subject having cancer comprising administering to the subject a therapeutically effective amount of a thioredoxin or a thioredoxin-like molecule and providing to the subject an anticancer therapy. In particular embodiments of the invention, the thioredoxin or thioredoxin-like molecule enhances the redox-cycling of the anticancer therapy thereby promoting cell killing.

Cancers contemplated in regard to the present invention may be premalignant, malignant or metastatic cancers. In further embodiments, the cancer may be a breast cancer, lung cancer, head and neck cancer, bladder cancer, bone cancer, bone marrow cancer, brain cancer, colon cancer, esophageal cancer, gastrointestinal cancer, gum cancer, kidney cancer, liver cancer, nasopharynx cancer, ovarian cancer, prostate cancer, skin cancer, stomach cancer, testis cancer, tongue cancer, or uterine cancer but is not limited to such. In particular embodiments, it is contemplated that the cancer is a breast cancer.

Any agent or therapeutic component that may have an increase in redox-cycling leading to sensitizing or enhancing or that promotes apoptosis due to thioredoxin or thioredoxin-like molecules is contemplated for use in the present invention. The anticancer therapy may comprise the administration of a chemotherapeutic agent such as, for example, an anthracycline in some preferred embodiments. Anthracyclines contemplated for use in the present invention include, but are not limited to, doxorubicin or daunomycin. In some embodiments of the invention, the anticancer therapy may comprise the administration of a radiotherapeutic agent such as, but not limited to, radiation. Administration of the thioredoxin or thioredoxin-like molecule may be before, at the same time as, or after the anticancer therapy. In further embodiments, the thioredoxin or thioredoxin-like molecule may be administered once or more than once. Compounds, agents or compositions of the present invention may be administered by any method known to one of ordinary skill in the art for administering a protein or gene therapy or a small molecule. The thioredoxin or thioredoxin-like molecule may be administered intravenously, intradermally intramuscularly, intraarterially, intralesionally, percutaneously, subcutaneously, or by an aerosol. In particular embodiments, the thioredoxin or thioredoxin-like molecules may be administered directly to a tumor.

In other particular embodiments, the anticancer therapy may be provided to a subject once or more than once. Administration of the anticancer therapy may be intravenously, intradermally, intramuscularly, intraarterially, intralesionally, percutaneously, subcutaneously, or by an aerosol.

In still another embodiment, the present invention may comprise delivering an expression construct comprising a nucleic acid encoding thioredoxin or thioredoxin-like molecule to a subject. The expression construct may be a viral vector such as, but not limited to, an adenoviral vector; an adeno-associated viral vector, a retroviral vector, a lentiviral vector, a herpes viral vector, polyoma viral vector or hepatitis B viral vector. In particular embodiments, the viral vector may be an adenoviral vector. The present invention further embodies a gene therapy such as adenoviral gene therapy, but is not limited to such.

In still yet a further embodiment, the present invention provides a method of sensitizing a cancer cell to an anticancer agent comprising providing an effective amount of a thioredoxin or a thioredoxin-like molecule and an anticancer therapy to the cancer cell. In still a flirther embodiment, the thioredoxin or thioredoxin-like molecule enhances the redox-cycling of the anticancer agent and/or induces apoptosis in the cancer cell. The cancer cell may be located in a tissue culture or in a subject. The subject may be a mammal such as a human.

In still a further particular embodiment of the invention, there is provided a method of ameliorating negative side-effects of an anticancer therapy in a subject comprising administering to the subject a therapeutically effective amount of a thioredoxin or a thioredoxin-like molecule and an anticancer therapy.

In still yet another particular embodiment of the invention, there is provided a pharmaceutical combination comprising a thioredoxin or a thioredoxin-like molecule and an anticancer agent. The anticancer agent may be a chemotherapeutic agent such as an anthracycline, but is not limited to such. The anthracycline may be doxorubicin or daunomycin but is not limited to such. The pharmaceutical combination may further comprise the thioredoxin or a thioredoxin-like molecule and the anticancer agent comprised together, each separately, in a pharmaceutically acceptable excipient.

An effective amount of compositions of the invention, generally, is defined as that amount sufficient to detectably and repeatedly ameliorate, reduce, minimize or limit the extent of the disease or its symptoms. More rigorous definitions may apply, including elimination, eradication or cure of the disease. An effective amount may be defined as that amount to sensitize, enhance, or promote apoptosis.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an, ” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Increased apoptosis of Trx9 cells in response to daunomycin. Vector or Trx9 cells were treated with daunomycin for 16 hr followed by detection of apoptosis using TUNEL assay. Upper panel, untreated vector and trx9 cells; lower panel, vector and Trx9 cells treated with daunomycin (1 μM, 16h).

FIGS. 2A-2C. FIG. 2A—Effect of thioredoxin overexpression on cell cycle distribution, apoptosis and cytochrome c release. Vector or Trx9 cells were treated with daunomycin (1 μM, 16 h) and cell cycle analysis was performed. Upper panel, histogram of cell cycle distribution of untreated vector and Trx9 cells; lower panel, histogram of cell cycle distribution of vector or Trx9 cells treated with daunomycin. Arrow in lower panel indicates “Sub-G1” peak. FIG. 2B—Data in sub-G1 peak in FIG. 2A is represented as a bar graph. FIG. 2C—Vector or Trx9 cells were treated with daunomycin as described for FIG. 2A, and cytochrome c release assay was performed as described in the method.

FIGS. 3A-3B. Effect of thioredoxin on daunomycin-mediated cell death. FIG. 3A—Cell proliferation assay was performed by BrdU ELISA. Vector or Trx9 cells were treated with daunomycin (1 μM, 16 h) and BrdU incorporation was determined. FIG. 3B—Cell death following treatment with daunomycin was determined by XTT assay, and the data was expressed as percent control of untreated cells.

FIG. 4. Effect of thioredoxin on clonogenic survival of vector or Trx9 cells treated with daunomycin. For determination of clonogenic survival, cells were treated with daunomycin and clonogenic survival was determined at the end of 14 days. Upper panel—untreated vector or Trx9 cells in 100 mm dishes. Lower panel—vector or Trx9 cells treated with 0.1 μM daunomycin.

FIGS. 5A-5C. Generation of O₂— in vector or Trx9 cells in response to daunomycin or doxorubicin. FIG. 5A—Vector or Trx9 cells were treated with daunomycin or doxorubicin and the generation of O₂— was determined by reduction of SOD-inhibitable cytochrome c. Data is presented as nmoles of O₂— produced per mg total cell protein. FIG. 5B—Fluorescent microscopy: vector or Trx9 cells were treated with daunomycin and were processed for DCF-DA assay. Upper panel—untreated vector or vector cells treated with doxorubicin; lower panel, untreated Trx9 cells or Trx9 cells treated with doxorubicin; inset—confocal microscopy showing most of the fluorescence in the cytosol. FIG. 5C—Effect of reductase inhibitors on O₂— generation. Trx9 cells were treated with various reductase inhibitors as indicated, and O₂— generation was assayed. Lane 1, Trx9 cells treated with 10 μM doxorubicin. Lane 2, Trx9 cells pre-incubated with 25 μM DPIC followed with 10 μM doxorubicin treatment; lane 3, Trx9 cells pre-incubated with 50 μM DPIC followed with 10 μM doxorubicin treatment; lane 4, Trx9 cells pre-incubated with 25 μM PAMP followed with 10 μM doxorubicin treatment; lane 5, Trx9 cells pre-incubated with 50 μM PAMP followed with 10 μM doxorubicin treatment; lane 6, Trx9 cells pre-incubated with 25 μM ABT followed with 10 μM doxorubicin treatment; lane 7, Trx9 cells pre-incubated with 50 μM PAMP followed with 10 μM doxorubicin treatment.

FIG. 6. Effect of thioredoxin overexpression on daunomycin-induced p53 or phospho-p53 (ser-15) expression. Vector or Trx9 were treated with 1 μM daunomycin for 16 hr followed by lysate preparation. p53 or phospho-p53 (ser-15) was detected. Lane 1, untreated vector cells; lane 2, untreated Trx9 cells; lane 3, vector treated with 1 μM daunomycin; lane 4, Trx9 cells treated with 1 μM daunomycin. Upper panel, p53; lower panel, phospho- p53 (ser-15).

FIGS. 7A-7B. Effect of thioredoxin overexpression on p53 DNA binding in response to daunomycin. FIG. 7A—Vector or Trx9 cells were treated with 1 μM daunomycin and nuclear extract was prepared and gel-shift assay was performed. Lane 1, untreated vector cells; lane 2, untreated Trx9 cells; lane 3, vector cells treated with daunomycin; lane 4, Trx9 cells treated with daunomycin. FIG. 7B—Effect of MnTBAP and DPIC on daunomycin-induced p53 DNA binding and p53 (ser-15) phosphorylation in Trx9 cells. Vector or Trx9 cells were treated with daunomycin either in the presence of MnTBAP (20 μM) or DPIC (50 μM) for 4 hr and gel-shift assay was performed. Additionally, total cell lysate 34 was prepared and phospho-p53 (ser-15) was determined using anti-phospho-p53 (ser-15) antibody. Lane 1, untreated Trx9 cells; lane 2, Trx9 cells treated with daunomycin (1 μM); lane 3, Trx 9 cells treated with 100 μM MnTBAP; lane 4, Trx9 cells treated with daunomycin+MnTBAP; lane 5, Trx9 cells treated with DPIC; lane 6, Trx9 cells treated with daunomycin+DPIC. Upper Panel; p53 gel-shift assay; lower panel, western blotting for phospho-p53(ser-15).

FIGS. 8A-8C. Effect of adenovirus-mediated overexpression of thioredoxin on daunomycin-induced p53 protein expression. FIG. 8A—MCF-7 cells were infected with pAdenoX, pAdenoX-Trx or pAdenoX-dnTrx. After 48 hr infected cells were treated with 1 μM daunomycin for 16 hr. Western analysis was performed. Lane 1, cells infected with pAdenoX; lane 2, cells treated with pAdenoX-Trx; lane 3, cells treated with pAdenoX-dnTrx; lanes 4-6, cells were infected with pAdenoX, pAdenoX-Trx or Adenox-dnTrx and treated with 1 μM daunomycin for 16 hr. FIG. 8B—MCF-7 cells were infected with 10-20 CFU of pAdenoX vector, pAdenox-Trx or pAdenox-dnTrx and Trx was determined using an ELISA assay (Das and White, 1998). The amount of Trx was expressed as picograms of Trx per mg protein. FIG. 8C—MCF-7 cells were infected with the viruses as described in FIG. 8B, and western analysis was performed using an anti-goat Trx antibody.

FIGS. 9A-9B. Proposed scheme for thioredoxin-mediated redox-cycling of anthracyclines. FIG. 9A—Redox-cycling of the quinone moiety of anthracyclines. The quinone is converted to a semiquinone radical by auto-oxidation in the presence of molecular oxygen and a flavoprotein enzyme (Fp). The reductive enzyme uses NADPH as the source of reducing equivalents. The model shows reduced thioredoxin (Trx-SH) acts as an electron donor for a bioreductive Fp enzyme that induces redox-cycling of the quinone moiety of anthracyclines. FIG. 9B—Anthracyclines can form complexes with cellular iron to form an anthracyline-iron complex (ANT-Fe^{2 +}), which can undergo redox-cycling in the presence of flavoprotein oxidoreductases. These reductases use NADPH to redox-cycle the ANT-Fe²⁺ complex. The model shows reduced thioredoxin can be a direct electron donor for a flavoprotein reductase. Additionally, thioredoxin can also provide electrons directly to the ANT-Fe²⁺ complex, which can reduce it to ANT-Fe³⁺, with the generation of superoxide radicals.

FIG. 10. Anthracyclines generate O₂— with the Trx system. O₂— generation was determined by the SOD-inhibitable reduction of ferricytochrome c. The reaction mixture contained the following: lane 1 NADPH blank, lane 2 3.2 lM Trx, lane 3 0.35 lg/ml TR, lane 4 3.2 lM Trx and 0.35 lg/ml TR, lane 5 10 lM daunomycin, lane 6 10 lM doxorubicin, lanes 7-12 the concentrations of Trx and TR were the same as those in lane 2 and lane 3 and the drug concentration was 10 lM. *P<0.05 vs lane 8, **P<0.05 vs lane 11.

FIGS. 11A-11C. FIG. 11A—Effect of thioredoxin concentration on O₂— generation with doxorubicin. O₂— generation was detected by SOD-inhibitable reduction of ferricytochrome c as described in the methods. Increasing the concentration of thioredoxin from 1.6 to 12.8 μM with constant TR (0.35 μg) and 10 μM doxorubicin enhances O₂— generation in a concentration dependant manner. FIG. 11B—Simultaneous detection of reduction of ferricytochrome c and disappearance of NADPH in 250 11 reactions adapted for a microplate containing increasing concentrations of Trx from 1.6 to 4.8 lM with constant TR (0.35 lg/ml) and 10 lM doxorubicin; d reduction of ferricytochrome c in the above reactions with SOD (5 U/ml). FIG 11C—Reduction of ferricytochrome c in the above reactions with SOD (5 U/ml).

FIGS. 12A-12B. FIG. 12A—Redox-cycling of anthracylines by thioredoxin system induce nicks in plasmid DNA mediated by O₂—. Plasmid DNA was incubated in the presence or absence of thioredoxin system as described in the methods. Lanes 1 and 4, Trx system only; lane 2 & 5, system with daunomycin or doxorubicin with SOD (10 U); lanes 3 and 6, Trx system with daunomycin or doxorubicin without SOD. FIG. 12B—Redox-cycling of anthracyclines by the Trx system induces nicks in plasmid DNA mediated by O₂—. Plasmid DNA pBLCAT6 incubated with doxorubicin and the Trx system for 2 h and analyzed by agarose gel electrophoresis. Lane 1 plasmid DNA (1 lg), lane 2 plasmid DNA+10 lM doxorubicin, lane 3 plasmid DNA+0.35 lg/ml TR, lane 4 plasmid DNA+TR+3.2 lM Trx, lane 5 plasmid DNA+TR+doxorubicin, lane 6 plasmid DNA+TR+Trx+doxorubicin, lane 7 plasmid DNA+TR+Trx+doxorubicin+SOD (2 U).

FIGS. 13A-13B. Oxidation of thioredoxin during redox-cycling of anthracyclines. FIG. 13A—Western blot detection of redox status of thioredoxin after carboxymethylation and separation in native PAGE was performed as described in the methods. Lane 1, E.coli Trx-SH standard (1 ng); lane 2, E.coli oxidized Trx standard; lane 3, Trx redox state in presence of E.coli Trx and TR; lane 4, Trx redox state in the presence of Trx, TR and doxorubicin; Trx redox state in the presence of Trx, TR, doxorubicin and SOD. FIG. 13B—Densitometric analysis of western blot shown in FIG. 13A.

FIGS. 14A-14B. Reduced thioredoxin donates electron to cytochrome P450 reductase. FIG. 14A—Cytochrome P450 reductase activity was determined as described in the methods. Lane 1, Blank, buffer and cytochrome c only; lane 2, 11.2 μM Trx-SH, NADPH (200 μM) and cytochrome P450 (10 nm); Lane 3, 11.2 μM Trx-S₂, NADPH (200 μM) and cytochrome P450 (10 nm). FIG. 14B—Reduced thioredoxin enhances redox-cycling of doxorubicin by reductases by donating electrons to cytochrome P450 reductase. Generation of superoxide was detected as reduction of SOD-inhibitable ferricytochrome c as described in the methods. Lane 1, 10 μM doxorubicin+11.2 μM E.coli Trx-SH; lane 2, cytochrome P450 reductase (0.04U)+11.2 μM Trx-SH; lane 3, cytochrome P450 reductase (0.04U)+11.2 μM E.coli Trx-SH+doxorubicin (10 μM); lane 4, 0.35 μg TR, 10 μM doxorubicin and 11.2 mM E.coli Trx-SH. * Significantly higher than blank or Trx-S₂ at p<0.05 level in FIG. 5A; ** significantly higher than lanes 1 or 2 at p<0.05 level in FIG. 14B.

FIGS. 15A-15B. Confocal microscopy of localization of E. coli in A549 cells using a monoclonal antibody against E. coli thioredoxin. A549 cells were incubated with or without E.coli Trx (32 μM) for 16 hours followed by detection of intracellular E.coli Trx using confocal microscopy as described in the method. FIG. 15A—A549 cells without E. coli Trx. FIG. 15B—A549 cells incubated with E. coli Trx (32 μM, 16 hr). Bright green fluorescence indicated the presence of E.coli Trx.

FIGS. 16A-16D. Externally added E. coli thioredoxin increases ROS generation in response to daunomycin. A549 lung adenocarcinoma cells were incubated with E. coli Trx (16 hr, 32 μM). Cells then were incubated with a 1 μM concentration of daunomycin and continuously monitored for DCF-DA fluorescence. FIG. 16A—A549 cells treated with E. coli Trx only. FIG. 16B—A549 cells treated with daunomycin only (1 μM). FIG. 16C—Cells treated with E. coli Trx (32 μM, 16 hr) and daunomycin (1 μM). Enhanced green fluorescence indicated increased ROS formation. Histogram towards left of each figure indicate the fluorescence of each cell, and thus the total fluorescence of the viewing area. FIG. 16D—Average DCF-DA fluorescence intensity of 100 cells in each experiment assayed in triplicate measured for a period of 300 s after the addition of doxorubicin.

FIGS. 17A-17E. A549 cells undergo increased DNA fragmentation in response to daunomycin in the presence of E. coli Trx. A549 cells were treated with a 32 μM of E.coli Trx for 16 hr. Following incubation, medium was changed and the cells were treated with a 1 μM of daunomycin for 4 hr. A TUNEL assay was performed as described in the methods. TUNEL positive nuclei were detected as green fluorescence on confocal microscopy. FIG. 17A—A549 cells. FIG. 17B—A549 cells treated with daunomycin (1 μM) for 4 hours. FIG. 17C—A549 cells treated with E.coli Trx (32 μM) for 16 hours. FIG. 17D—A549 cells treated with E.coli Trx (32 μM) for 16 hours followed by treatment with daunomycin (1 μM) for 4 hours. FIG. 17E—Graph representing percent TUNEL positive nuclei in cells treated with daunomycin (1 lM) with or without E. coli Trx (32 lM). **Significantly higher than cells treated with anthracycline only.

FIG. 18. Enhanced p53 DNA binding in E. coli Trx-enriched A549 cells in response to daunomycin. A549 cells were incubated with 32 lM E. coli Trx for 16 h. Following incubation cells were treated with daunomycin (1 lM) for 4 h, nuclear extract was prepared and EMSA was performed as described in Materials and methods. Lane 1 control A549 cells, lane 2 cells incubated with 32 lM E. coli Trx (16 h), lane 3 cells treated with 1 lM daunomycin, lane 4 cells incubated with E. coli Trx followed by treatment with 1 lM daunomycin.

### DESCRIPTION OF ILLUSTRATIVE EMBODIMENTS

I. The Present Invention

The present invention relates to the discovery that increasing the expression of a small cellular redox protein thioredoxin (Trx) sensitizes human breast cancer cells to enhanced apoptotic action of the anthracycline class of anticancer drugs. MCF-7 cells stably overexpressing thioredoxin are more cytotoxic and apoptotic in response to daunomycin. Additionally, thioredoxin-overexpressing MCF-7 cells are not able to propagate into colonies in response to daunomycin as compared to vector control cells. Thioredoxin increases the level of superoxide anion (O₂—) generation in anthracycline-treated cell extracts. Enhanced generation of O₂— in response to daunomycin in thioredoxin-overexpressing MCF-7 cells is inhibited by DPIC, a general NADPH reductase inhibitor, demonstrating that thioredoxin provides reducing equivalents to a bioreductive enzyme for redox-cycling of daunomycin. Additionally, thioredoxin increases p53 DNA binding and protein expression in response to anthracyclines. In particular embodiments of the invention, thioredoxin increases apoptosis of breast cancer cells by increasing the redox-cycling of the anthracycline class of antitumor drugs.

The present invention also demonstrates that E. coli thioredoxin can enhance the redox-cycling of anthracyclines, and thus generates O₂—. Additionally, reduced thioredoxin can also act as an electron donor for cytochrome P450 reductase. E.coli thioredoxin is also capable of redox-cycling anthracyclines in lung adenocarcinoma A549 cells.

Thus, the present invention provides thioredoxin as a therapeutic agent for sensitizing cancer cells to anticancer therapy, such as but not limited to, anthracycline chemotherapy.

II. Thioredoxin or Thioredoxin-like Molecules

The present invention employs a thioredoxin or thioredoxin-like peptide, polypeptide, protein, or compositions thereof for treating neoplastic diseases in combination with an anticancer therapy such as an anthracycline.

The present invention provides purified, and in preferred embodiments, substantially purified mammalian thioredoxin or thioredoxin-like proteins, polypeptides, or peptides. The term “purified mammalian thioredoxin or thioredoxin-like proteins, polypeptides, or peptides” as used herein, is intended to refer to a thioredoxin or thioredoxin-like proteinaceous composition, isolatable from mammalian cells or recombinant host cells, wherein the thioredoxin or thioredoxin-like protein, polypeptide, or peptide is purified to any degree relative to its naturally-obtainable state, i.e., relative to its purity within a cellular extract. A purified thioredoxin or thioredoxin-like protein, polypeptide, or peptide therefore also refers to a wild-type or mutant thioredoxin or thioredoxin-like protein, polypeptide, or peptide free from the environment in which it naturally occurs. Human thioredoxin has a sequence of SEQ ID NO:2, while E. coli thioredoxin has a sequence of SEQ ID NO:4, monkey thioredoxin has a sequence of SEQ ID NO:6 and rat thioredoxin has a sequence of SEQ ID NO:8.

The thioredoxin or thioredoxin-like proteins of the present invention may be full length proteins, such as being about 105 or about 109 or about 166 amino acids in length. The thioredoxin or thioredoxin-like proteins, polypeptides and peptides may also be less than full length proteins, such as individual polypeptides, domains, regions or even epitopic peptides. Where less than full length thioredoxin or thioredoxin-like proteins are concerned, the most preferred will be those containing predicted immunogenic sites and those containing the functional domains identified herein.

Encompassed by the invention are proteinaceous segments of relatively small peptides, such as, for example, peptides of from about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 40, about 45, to about 50, about 60, about 70, about 80, about 90, about 100, about 110, about 120, about 130, about 140, about 150, about 160, about 170 or more amino acids in length, and more preferably, of from about 105 to about 170 amino acids in length; as set forth in SEQ ID NO:2, SEQ ID NO:4, SEQ ID NO:6 or SEQ ID NO:8.

Generally, “purified” will refer to a thioredoxin or thioredoxin-like protein, polypeptide, or peptide composition that has been subjected to fractionation to remove various non- thioredoxin or thioredoxin-like protein, polypeptide, or peptide, and which composition substantially retains its thioredoxin or thioredoxin-like activity, as may be assessed by methods described herein or would be known to one of skill in the art.

Where the term “substantially purified” is used, this will refer to a composition in which the thioredoxin or thioredoxin-like protein, polypeptide, or peptide forms the major component of the composition, such as constituting about 50% or more of the proteinaceous molecules in the composition. In preferred embodiments, a substantially purified proteinaceous molecule will constitute more than 60%, 70%, 80%, 90%, 95%, 99% or even more of the proteinaceous molecules in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of thioredoxin or thioredoxin-like proteins, polypeptides, or peptides will be known to those of skill in the art in light of the present disclosure. These include, for example, determining the specific proteinaceous molecule's activity of a fraction, or assessing the number of proteins, polypeptides and peptides within a fraction by gel electrophoresis. Assessing the number of proteinaceous molecules within a fraction by SDS/PAGE analysis will often be preferred in the context of the present invention as this is straightforward.

To purify a thioredoxin or thioredoxin-like protein, polypeptide, or peptide a natural or recombinant composition comprising at least some thioredoxin or thioredoxin-like proteins, polypeptides, or peptides will be subjected to fractionation to remove various non- thioredoxin or thioredoxin-like components from the composition. In addition to those techniques described in detail herein below, various other techniques suitable for use in purification of a proteinaceous molecule will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques. Another example is the purification of a thioredoxin or thioredoxin-like fusion protein using a specific binding partner. Such purification methods are routine in the art.

The exemplary purification methods disclosed herein represent exemplary methods to prepare a substantially purified thioredoxin or thioredoxin-like protein, polypeptide, or peptide. These methods are preferred as they result in the substantial purification of the thioredoxin or thioredoxin-like protein, polypeptide or peptide in yields sufficient for further characterization and use. However, given the nucleic acids and proteinaceous molecules provided by the present invention, any purification method may be employed.

Although preferred for use in certain embodiments, there is no general requirement that the thioredoxin or thioredoxin-like protein, polypeptide, or peptide always be provided in their most purified state. Indeed, it is contemplated that less substantially purified thioredoxin or thioredoxin-like protein, polypeptide or peptide, which are nonetheless enriched in thioredoxin or thioredoxin-like proteinaceous compositions, relative to the natural state, will have utility in certain embodiments. These include, for example, antibody generation where subsequent screening assays using purified thioredoxin or thioredoxin-like proteinaceous molecules are conducted.

Methods exhibiting a lower degree of relative purification may have advantages in total recovery of proteinaceous molecule product, or in maintaining the activity of an expressed proteinaceous molecule. Inactive products also have utility in certain embodiments, such as, e.g., in antibody generation.

A. Nucleic Acids Encoding Thioredoxin or Thioredoxin-like Molecule

Important aspects of the present invention concern isolated DNA segments encoding wild-type, polymorphic or mutant thioredoxin or thioredoxin-like molecule such as proteins, polypeptides or peptides thereof, comprising the sequence of SEQ ID NO:1, SEQ ID NO:3, SEQ ID NO:5 or SEQ ID NO:7, and biologically functional equivalents thereof.

The present invention concerns DNA segments, isolatable from mammalian cells, such as mouse or human cells, that are free from total genomic DNA and that are capable of expressing a protein, polypeptide or peptide that is a thioredoxin or thioredoxin-like molecule. As used herein, the term “DNA segment” refers to a DNA molecule that has been isolated free of total genomic DNA of a particular species. Therefore, a DNA segment encoding a thioredoxin or thioredoxin-like molecule refers to a DNA segment that contains wild-type, polymorphic or mutant thioredoxin coding sequences yet is isolated away from, or purified free from, total mammalian genomic DNA. Included within the term “DNA segment”, are DNA segments and smaller fragments of such segments, and also recombinant vectors, including, for example, plasmids, cosmids, phage, viruses, and the like.

Similarly, a DNA segment comprising an isolated or purified thioredoxin or a thioredoxin-like gene refers to a DNA segment including a thioredoxin or thioredoxin-like protein, polypeptide or peptide coding sequences and, in certain aspects, regulatory sequences, isolated substantially away from other naturally occurring genes or protein encoding sequences. In this respect, the term “gene” is used for simplicity to refer to a functional protein, polypeptide or peptide encoding unit. As will be understood by those of skill in the art, this functional term includes both genomic sequences, cDNA sequences and engineered segments that express, or may be adapted to express, proteins, polypeptides, domains, peptides, fusion proteins and mutants of thioredoxin or a thioredoxin-like molecule encoded sequences.

The present invention contemplates the use of a nucleic acid(s) encoding a thioredoxin or thioredoxin-like molecule such as a peptide, polypeptide, or protein. A “nucleic acid” as used herein includes single-stranded and double-stranded molecules, as well as DNA, RNA, chemically modified nucleic acids and nucleic acid analogs. It is contemplated that a nucleic acid within the scope of the present invention may be of about 10, about 15, about 20, about 25, about 30, about 35, about 40, about 45, about 50, about 55, about 60, about 65, about 70, about 75, about 80, about 85, about 90, about 95, about 100 to about 200, of about 210 to about 250, of about 260 to about 300, of about 310 to about 350, of about 360 to about 400, of about 410 to about 450, of about 460 to about 500, of about 510 to about 550, of about 560 to about 600, of about 610 to about 650, of about 660 to about 700, of about 710 to about 750, of about 760 to about 800, of about 810 to about 850, of about 860 to about 900, about 1000, about 1100, about 1200, about 1300, about 1400, about 1500, about 1750, about 2000, about 2250, about 2500 or greater nucleotide residues in length. Those of skill will recognize that in cases where the nucleic acid region encodes a thioredoxin or a thioredoxin-like molecule such as a peptide, polypeptide or protein or mutant or mimetic thereof, the nucleic acid region can be quite long, depending upon the number of amino acids in the thioredoxin or thioredoxin-like molecule.

“Isolated substantially away from other coding sequences” means that the gene of interest, in this case a thioredoxin or thioredoxin-like gene, forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

In particular embodiments, the invention concerns isolated DNA segments that encode a thioredoxin or thioredoxin-like protein, polypeptide or peptide that includes within its amino acid sequence a contiguous amino acid sequence in accordance with, or essentially as set forth in, SEQ ID NO:2, SEQ ID NO:4, SEQ ID NO:6 or SEQ ID NO:8.

The term “a sequence essentially as set forth in SEQ ID NO:2, SEQ ID NO:4, SEQ ID NO:6 or SEQ ID NO:8” means that the sequence substantially corresponds to a portion of SEQ ID NO:2, SEQ ID NO:4, SEQ ID NO:6 or SEQ ID NO:8 and has relatively few amino acids that are not identical to, or a biologically functional equivalent of, the amino acids of SEQ ID NO:2, SEQ ID NO:4, SEQ ID NO:6 or SEQ ID NO:8.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99% of amino acids that are identical or functionally equivalent to the amino acids of SEQ ID NO:2, SEQ ID NO:4, SEQ ID NO:6 or SEQ ID. NO:8 will be sequences that are “essentially as set forth in SEQ ID NO:2, SEQ ID NO:4, SEQ ID NO:6 or SEQ ID NO:8”, provided the biological activity of the protein is maintained.

In certain other embodiments, the invention concerns isolated DNA segments that include within their sequence a nucleic acid sequence essentially as set forth in SEQ ID NO:1, SEQ ID NO:3, SEQ ID NO:5 or SEQ ID NO:7. The term “essentially as set forth in SEQ ID NO:1, SEQ ID NO:3, SEQ ID NO:5 or SEQ ID NO:7” is used in the same sense as described above and means that the nucleic acid sequence substantially corresponds to a portion of SEQ ID NO:1, SEQ ID NO:3, SEQ ID NO:5 or SEQ ID NO:7, and has relatively few codons that are not identical, or functionally equivalent, to the codons of SEQ ID NO:l, SEQ ID NO:3, SEQ ID NO:5 or SEQ ID NO:7. Again, DNA segments that encode proteins, polypeptide or peptides exhibiting thioredoxin or a thioredoxin-like molecule will be most preferred.

The term “functionally equivalent codon” is used herein to refer to codons that encode the same amino acid, such as the six codons for arginine and serine, and also refers to codons that encode biologically equivalent amino acids. For optimization of expression of thioredoxin or a thioredoxin-like molecule in human cells, the codons are shown in Table 1 in preference of use from left to right. Thus, the most preferred codon for alanine is thus “GCC”, and the least is “GCG” (see Table 1 below). Codon usage for various organisms and organelles can be found on the internet at the Codon Usage Database website, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), a eukaryote (e.g., a protist, a plant, a fungi, an animal), a virus and the like, as well as organelles that contain nucleic acids, such as mitochondria or chloroplasts, based on the preferred codon usage as would be known to those of ordinary skill in the art.

TABLE 1
Preferred Human DNA Codons

| Amino Acids | | | Codons | | | | | |
|---|---|---|---|---|---|---|---|---|
| Alanine | Ala | A | GCC | GCT | GCA | GCG | | |
| Cysteine | Cys | C | TGC | TGT | | | | |
| Aspartic acid | Asp | D | GAC | GAT | | | | |
| Glutamic acid | Glu | E | GAG | GAA | | | | |
| Phenylalanine | Phe | F | TTC | TTT | | | | |
| Glycine | Gly | G | GGC | GGG | GGA | GGT | | |
| Histidine | His | H | CAC | CAT | | | | |
| Isoleucine | Ile | I | ATC | ATT | ATA | | | |
| Lysine | Lys | K | AAG | AAA | | | | |
| Leucine | Leu | L | CTG | CTC | TTG | CTT | CTA | TTA |
| Methionine | Met | M | ATG | | | | | |
| Asparagine | Asn | N | AAC | AAT | | | | |
| Proline | Pro | P | CCC | CCT | CCA | CCG | | |
| Glutamine | Gln | Q | CAG | CAA | | | | |
| Arginine | Arg | R | CGC | AGG | CGG | AGA | CGA | CGT |
| Serine | Ser | S | AGC | TCC | TCT | AGT | TCA | TCG |
| Threonine | Thr | T | ACC | ACA | ACT | ACG | | |
| Valine | Val | V | GTG | GTC | GTT | GTA | | |
| Tryptophan | Trp | W | TGG | | | | | |
| Tyrosine | Tyr | Y | TAC | TAT | | | | |

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein, polypeptide or peptide activity where an amino acid sequence expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

Excepting intronic or flanking regions, and allowing for the degeneracy of the genetic code, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99% of nucleotides that are identical to the nucleotides of SEQ ID NO:1, SEQ ID NO:3, SEQ ID NO:5 or SEQ ID NO:7 will be sequences that are “essentially as set forth in SEQ ID NO:1, SEQ ID NO:3, SEQ ID NO:5 or SEQ ID NO:7”.

One may also prepare fusion proteins, polypeptides and peptides, e.g., where the thioredoxin or thioredoxin-like proteinaceous material coding regions are aligned within the same expression unit with other proteins, polypeptides or peptides having desired functions, such as for purification or immunodetection purposes (e.g., proteinaceous compositions that may be purified by affinity chromatography and enzyme label coding regions, respectively).

Encompassed by the invention are DNA segments encoding relatively small peptides, such as, for example, peptides of from about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 40, about 45, to about 50 about 55, about 60, about 65, about 70, about 75, about 80, about 85, about 90, about 95, about 100, about 110, about 120, about 125, about 130 or more amino acids in length, and more preferably, of from about 105 to about 170 amino acids in length; as set forth in SEQ ID NO:2, SEQ ID NO:4, SEQ ID NO:6, or SEQ ID NO:8 and also larger polypeptides up to and including proteins corresponding to the full-length sequences set forth in SEQ ID NO:2, SEQ ID NO:4, SEQ ID NO:6, or SEQ ID NO:8, and any range derivable therein and any integer derivable range.

In addition to the “standard” DNA and RNA nucleotide bases, modified bases are also contemplated for use in particular applications of the present invention. Modified bases are well known to one of ordinary skill in the art.

B. Vectors and Expression Constructs

Where incorporation into an expression vector is desired, a nucleic acid encoding a thioredoxin or thioredoxin-like peptide, polypeptide or protein may also comprise a natural intron or an intron derived from another gene. It is contemplated in the present invention, that virtually any type of vector may be employed in any known or later discovered method to deliver nucleic acids encoding a thioredoxin or thioredoxin-like peptide, polypeptide or protein; or constructs thereof, including mutants and mimetic thereof. Such vectors may be viral or non-viral vectors as described herein, and as are known to those skilled in the art. An expression construct comprising a nucleic acid encoding a thioredoxin or thioredoxin-like peptide, polypeptide, or protein thereof may comprise a virus or engineered construct derived from a viral genome.

The ability of certain viruses to enter cells via receptor-mediated endocytosis and to integrate into the host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986) and adeno-associated viruses. Retroviruses also are attractive gene transfer vehicles (Nicolas and Rubenstein, 1988; Temin, 1986) as are vaccina virus (Ridgeway, 1988) and adeno-associated virus (Ridgeway, 1988). Such vectors may be used to (i) transform cell lines in vitro for the purpose of expressing proteins of interest or (ii) to transform cells in vitro or in vivo to provide therapeutic polypeptides for gene therapy such as thioredoxin or thioredoxin-like peptides, polypeptides, proteins or mutants or mimetics thereof.

III. Anticancer Therapy

In particular embodiments, the present invention contemplates the use of an anticancer therapeutic agent for the treatment and/or prevention of cancer. The anticancer therapy contemplated for use with the thioredoxin or thioredoxin-like. molecules of the present invention employs anthracycline therapy and/or radiotherapy. Both anthracyclines and radiotherapy are known in the art to exert their cytotoxicity by producing oxygen-related free radicals, which imposes oxidative stress to the cancer cell leading to apoptosis. This effect may be further enhanced by thioredoxin or thioredoxin-like molecules that maintain cellular redox status.

A. Anthracyclines

Anthracyclines comprise a class of antitumor drugs that undergo redox-cycling in living cells. This produces increased amounts of reactive oxygen species and semiquinone radicals, both of which can cause DNA damage, and consequently trigger p53-mediated apoptotic death of cancer cells. Anthracyclines have been used effectively in the clinic, as chemotherapeutic agents for treating a variety of cancers including breast cancer (Gluck, 2002; Weiss, 1992). However, their use has been limited by acute and chronic cardiotoxicity at doses that are effective for cancer chemotherapy (Mordente et al., 2001; Hrdina et al., 2000; Childs et al., 2002). The mechanism of action of these drugs is attributed to the intercalation of the drug into DNA, and generation of reactive oxygen species (ROS) that can cause DNA damage leading to apoptotic cell death (Gutierrez, 2000; Gutierrez et al., 1983; Keizer et al., 1990; Bolton et al., 2000).

In cancer cells, anthracyclines have been shown to induce p53-dependent apoptosis (Asher et al., 2001; Cho, 1999). Anthracyclines also cause DNA damage, which increases p53 expression (Ngo et al., 1998; Sadji-Ouatas et al., 2002). p53 is a sequence-specific transcription factor, which can induce pro-apoptotic or suppress anti-apoptotic genes in response to DNA damage or irreparable cell cycle arrest (Maya et al., 2001). Phosphorylation of p53 at ser-15 residue dissociates MDM2 and activates p53 as a transcription factor, which binds to various p53-dependent genes resulting in their activation or repression (Maya et al., 2001).

Anthracyclines contain quinone moieties in their structure, which can undergo biochemical reduction by one or two electrons that is catalyzed by flavoenzymes in the cell using NADPH as an electron donor (Iyanagi and Yamazaki, 1969; Iyanagi and Yamazaki, 1970). This bio-reductive process generates semiquinone radicals with concomitant production of superoxide anion (O₂—). The semiquinone radicals intercalate with the DNA causing DNA damage. The formation of O₂— is the beginning of a cascade that generates hydrogen peroxide and hydroxyl radicals, generally referred to as reactive oxygen species (Mordente et al., 2001). In addition to various bioreductive enzymes, low molecular weight protein or non-protein thiols may also take part in the redox cycling process (Hrdina et al., 2000).

The present invention contemplates the use of any anthracycline or analog thereof, known to one of ordinary skill in the art. In particular embodiments, anthracyclines contemplated for use in the present invention include daunomycin and doxorubicin, but the invention is not limited to such anthracyclines.

1. Doxorubicin

Doxorubicin hydrochloride, 5,12-Naphthacenedione, (8 s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis and mitosis, and promotes chromosomal aberrations.

Administered alone, it is the drug of first choice for the treatment of thyroid adenoma and primary hepatocellular carcinoma. It is a component of first-choice in combination with other agents for the treatment of ovarian tumors, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Since doxorubicin is poorly absorbed it is administered intravenously. The pharmacokinetics of this chemotherapeutic agent are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, for an adult, administered intravenously, are 60 to 75 mg/m² at 21-day intervals, or 25 to 30 mg/m² on each of 2 or 3 successive days repeated at 3- or 4-wk intervals, or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk may be administered. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m²², 300 mg/m², 350 mg/m², 400 mg/m², 425 mg/m², 450 mg/m², 475 mg/m₂, 500 mg/m². Of course, all of these dosages are exemplary, and any dosage in-between these points is also expected to be of use in the present invention.

2. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine or daunomycin, is also widely used as an therapeutic agent for treating a variety of cancers. Daunorubicin intercalates into DNA, blocks DNA-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. As oral absorption is poor, this drug is administered intravenously. The half-life of distribution of daunorubicin is 45 min, and the half-life of elimination, about 19 hr. The half-life of its active metabolite, daunorubicinol, is about 27 hr. Daunorubicin is metabolized mostly in the liver and also secreted into the bile (ca 40%).

Suitable doses (base equivalent), administered intravenously to an adult, younger than 60 yrs, are 45 mg/m²/day (30 mg/m² for patients older than 60 yrs) for 1, 2 or 3 days every 3 or 4 wk, or 0.8 mg/kg/day for 3 to 6 days every 3 or 4 wk; no more than 550 mg/m² should be given in a lifetime, except only 450 mg/m² if there has been chest irradiation. For children, a suitable dose that may be administered is 25 mg/m² once a week unless the age is less than 2 yrs or the body surface less than 0.5 m, in which case the weight-based adult schedule is used. Daunorubicin is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the present invention.

3. Other Anthracylcine Agents

Any anthracycline or derivative thereof that may undergo redox-cycling is contemplated for use as a therapeutic agent in the present invention. Other anthracycline agents that may be employed in the present invention may include, but are not limited to, aclarubicin, epirubicin, idarubicin or mitoxantrone (mitozantrone) an anthracycline derivative. Anthracyclines, as in the present invention, may include any derivative known to one of ordinary skill in the art, for examples see U.S. Pat. Nos. 4,737,583; 4,316,011; 3,988,315; 4,039,736; 4,127,714; 4,144,329; 4,169,142; and U.S. Patent Applications 20030125268; 20030023052; 20020137694; and 20010053845 each incorporated herein by reference.

i. Epirubicin

Epirubicin is a stereoisomer of doxorubicin in which the hydroxyl group in the C-4′ position of the amino sugar is epimerized. Like other anthracyclines, the precise mechanism of action of epirubicin is unknown, but is primarily related to intercalation of the planar ring with DNA and subsequent inhibition of DNA and RNA synthesis. Epirubicin is cell cycle phase-nonspecific. Clinical trials suggest that it is as effective as doxorubicin in the treatment of breast cancer. Epirubicin has been used in the treatment of gastric cancer, Hodgkin's disease, lung cancer non-Hodgkin's lymphoma, ovarian cancer, pancreatic cancer sarcoma, and soft tissue sarcoma. A maximum cumulative dose of 0.9 to 1 lg/m² is recommended to help avoid cardiotoxicity. Like doxorubicin it is given intravenously and by bladder instillation.

ii. Aclarubicin and Idarubicin

Aclarubicin and idarubicin belong to the anthracycline class of antineoplastic agents with general properties similar to those of doxorubicin or daunorubicin. Idarubicin is an anthracycline analogue of daunorubicin. It is 5 to 6 times more potent and less cardiotoxic than daunorubicin. The mechanism of action of these anthracyclines is poorly understood. Cytotoxicity is generally attributed to intercalation of the drug into DNA and/or inhibition of DNA topoisomerase II activity resulting in double and single strand DNA breaks. Aclarubicin and idarubicin are usually given intravenously but may also be given orally.

iii. Mitoxantrone

Mitoxantrone (mitozantrone) is an anthracenedione structurally similar to doxorubicin. Its exact mechanism of action is unknown but includes intercalation with DNA to cause inter/intrastrand cross-linking. It also causes DNA strand breaks through binding with the phosphate backbone of DNA. Mitoxantrone is cell cycle phase-nonspecific. Mitoxantrone it is used for the treatment of metastatic breast cancer but has also been used for treating gastric, liver, ovarian and prostate cancers. This anthracycline agent is also licensed for use in the treatment of non-Hodgkin's lymphoma and adult non-lymphocytic leukaemia. It is given intravenously and is well tolerated but myelosuppression and dose-related cardiotoxicity has been known to occur; cardiac examinations are recommended after a cumulative dose of 160 mg/m².

B. Radiotherapy

Radiotherapy, also called radiation therapy, involves the use of ionizing radiation to treat cancers and other diseases. Ionizing radiation deposits energy that injures or destroys cells in the area being treated (the “target tissue”) by damaging their genetic material, and thereby inhibiting cell proliferation. Ionizing radiation induces the formation of hydroxyl radicals, placing the cells under oxidative stress. These radicals damage DNA, which causes cytotoxicity.

Radiotherapeutic agents that cause DNA damage are well known in the art and have been extensively used. Radiotherapeutic agents, through the production of oxygen-related free radicals and DNA damage, may lead to cell death or apoptosis. These agents may include, but are not limited to, γ-rays, X-rays, and/or the directed delivery of radioisotopes to tumor cells (known as internal radiotherapy). Internal radiotherapy may further include but is not limited to, brachytherapy, interstitial irradiation, and intracavitary irradiation. Other radiotherapeutic agents that are DNA damaging factors include microwaves and UV-irradiation. These factors effect a broad range of damage on DNA, on the precursors of DNA, on the replication and repair of DNA, and on the assembly and maintenance of chromosomes.

Other approaches to radiation therapy are also contemplated in the present invention. Such techniques may comprise intraoperative irradiation, in which a large dose of external radiation is directed at the tumor and surrounding tissue during surgery; and particle beam radiation therapy which involves the use of fast-moving subatomic particles to treat localized cancers. Radiotherapy may further involve the use of radiosensitizers and/or radioprotectors to increase the effectiveness of radiation therapy. Radiolabeled antibodies may also be used to deliver doses of radiation directly to the cancer site, this is known as radioimmunotherapy.

Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Thus, the present invention contemplates the use of radiotherapeutic agents for treating localized solid tumors, such as cancers of the skin, tongue, larynx, brain, breast, or uterine cervix, but is not limited to such. Radiotherapy may also be used to treat leukemia and lymphoma (cancers of the blood-forming cells and lymphatic system, respectively. Internal radiotherapy is frequently used for cancers of the tongue, uterus, and cervix.

IV. Combinations of Thioredoxin or Thioredoxin-like Molecules and Anticancer Agents

The compounds and methods of the present invention may be used in the context of treating or preventing neoplastic diseases/conditions including but not limited to cancer or hyperplasia. In order to increase the effectiveness of an anticancer agent in treating and/or preventing cancer, the present invention contemplates administering an anticancer agent, as described above, in combination with a thioredoxin or thioredoxin-like molecules to a subject having cancer. By increasing the redox-cycling of an anticancer agent, the thioredoxin or thioredoxin-like molecule enhances its effectiveness as a therapeutic agent by sensitizing cancer cells to cell killing agents. Thus, in particular embodiments, the present invention contemplates the use of a thioredoxin or thioredoxin-like molecules in combination with an anthracycline agent or a radiotherapeutic agent, or both a radiotherapeutic and an anthracycline agent for the treatment or prevention of cancer.

For example, the treatment of a cancer may be implemented with thioredoxin or thioredoxin-like compounds and an anthracylcine therapeutic compound such as, but not limited to, doxorubicin, or daunorubicin, or an analog or derivative thereof. In other embodiments of the invention, the thioredoxin or thioredoxin-like molecule may be administered with a radiotherapeutic agent such as, but not limited to, x-rays, γ-rays or radioisotopes.

The terms “contacted” and “exposed,” when applied to a cell, are used herein to describe the process by which a therapeutic construct and, for example, a anthracycline or radiotherapeutic agent are delivered to a target cell or are placed in direct juxtaposition with the target cell. To achieve cell killing or stasis, both agents are delivered to a cell in a combined amount effective to kill the cell or prevent it from dividing.

Various combinations of times of treatment may be used in the present invention. For example, therapies involving thioredoxin or thioredoxin-like molecules may precede or follow that of an anthracycline or radiotherapeutic agent by intervals ranging from minutes to weeks. Where anthracycline or radiotherapeutic agents, and thioredoxin or thioredoxin-like molecules are provided or administered separately to the subject, one would generally ensure that a significant period of time did not expire between the time of each delivery, such that the anthracycline or radiotherapeutic agent and thioredoxin or thioredoxin-like molecules would still be able to exert an advantageously combined effect on the subject. In such instances, it is contemplated that one may provide or administer to the subject any of the agents within about 1-6 or about 12-24 hr of each other and, more preferably, within about 6-12 hr of each other, with a delay time of only about 12 hr being most preferred. In some situations, it may be desirable to extend the time period for treatment significantly, however, where several days (2, 3, 4, 5, 6, or 7) to several weeks (1, 2, 3, 4, 5, 6, 7 or 8) lapse between the respective administrations.

It also is conceivable that more than one administration of either the thioredoxin or thioredoxin-like molecules and/or anthracycline and/or radiotherapeutic agent will be desired. Various combinations may be employed, where the thioredoxin or thioredoxin-like molecules is “A” and an anticancer therapy such as an anthracycline or radiotherapeutic agent is “B”, as exemplified below:

A/B/A B/A/B B/B/A A/A/B B/A/A A/B/B B/B/B/A
B/B/A/B A/A/B/B A/B/A/B A/B/B/A B/B/A/A
B/A/B/A B/A/A/B B/B/B/A A/A/A/B B/A/A/A
A/B/A/A A/A/B/A A/B/B/B B/A/B/B B/B/A/B

Administration of the thioredoxin or thioredoxin-like molecules and an anthracycline or radiotherapeutic of the present invention to a subject will follow general protocols for the administration of that particular anthracycline or radiotherapeutic agent, taking into account the toxicity, if any, of the thioredoxin or thioredoxin-like molecules in combination with an anthracycline or radiotherapeutic composition used for treatment. It is expected that the treatment cycles would be repeated as necessary.

V. Adjunct Therapies For Use In Conjunction With Thioredoxin or Molecules Thereof and Anticancer Therapies

In other embodiments, the present invention contemplates the use of a composition comprising a thioredoxin or thioredoxin-like molecule and an anticancer therapeutic compound(s) in conjunction with an adjunct therapy, for treating or preventing cancers or hyperplasias. An adjunct therapy, as contemplated in the present invention, is any agent capable of negatively affecting cancer in a subject. Such agents may be effective in the treatment of neoplastic diseases and conditions. Adjunct therapies may include, but are not limited to, biological agents (biotherapy), chemotherapy agents, or surgical intervention, or any such therapies as is known to those of skill in the art.

In the context of the present invention, it is contemplated that the composition of a thioredoxin or thioredoxin-like molecule and an anthracycline agent may be used in conjunction with an adjunct therapeutic. In other embodiments, the composition comprising a thioredoxin or thioredoxin-like molecule and a radiotherapeutic agent may be used in conjunction with an adjunct therapeutic. In further embodiments of the invention, the composition comprising a thioredoxin or thioredoxin-like molecule, an anthracycline agent and a radiotherapeutic agent may be used in conjunction with an adjunct therapeutic. It is also contemplated that one or more adjunct therapies, as is known to one of ordinary skill in the art, may be employed with the thioredoxin or thioredoxin-like molecule and anthracycline or radiotherapy composition(s) as described herein.

Adjunct therapies for use in combination with the composition comprising thioredoxin or thioredoxin-like molecules and an anticancer therapy may precede or follow that of the adjunct therapy or the anticancer therapy by intervals ranging from minutes to weeks. Where the adjunct agent(s), and the composition of a thioredoxin or thioredoxin-like molecule and an anticancer therapy are provided or administered separately to the subject, one would generally ensure that a significant period of time did not expire between the time of each delivery, such that the adjunct agent and the composition of thioredoxin or thioredoxin-like molecules in combination with the anticancer compound would still be able to exert an advantageously combined effect on the subject. It is contemplated that any of these agents may be provided or administered to a subject within about 1-6 or about 12-24 hr of each other and, more preferably, within about 6-12 hr of each other, with a delay time of only about 12 hr being most preferred. In some situations, it may be desirable to extend the time period for treatment significantly, however, where several days (2, 3, 4, 5, 6, or 7) to several weeks (1, 2, 3, 4, 5, 6, 7 or 8) lapse between the respective administrations.

It also is conceivable that more than one administration of either the composition of a thioredoxin or thioredoxin-like molecule and the anticancer agent and/or adjunct agent will be desired. Various combinations may be employed, where the composition of a thioredoxin or thioredoxin-like molecule in combination with an anticancer agent is “C” and the adjunct agent is “D”, as exemplified below:

C/D/C D/C/D D/D/C C/C/D D/C/C C/D/D D/D/D/C
D/D/C/D C/C/D/D C/D/C/D C/D/D/C D/D/C/C
D/C/D/C D/C/C/D D/D/D/C C/C/C/D D/C/C/C
C/D/C/C C/C/D/C C/D/D/D D/C/D/D D/D/C/D

Administration of the composition of a thioredoxin or thioredoxin-like molecule in combination with an anticancer agent of the present invention and an adjunct agent to a subject will follow general protocols for the administration of that particular adjunct therapy, taking into account the toxicity, if any, of the composition of a thioredoxin or thioredoxin-like molecule in combination with the anticancer agent or adjunct agent used for treatment. It is expected that the treatment cycles would be repeated as necessary.

1. Chemotherapy

An adjunct therapy contemplated in the present invention is chemotherapy. Chemotherapeutic agents may include, for example, anthracyclines, cisplatin (CDDP), carboplatin, procarbazine, mechlorethamine, cyclophosphamide, camptothecin, ifosfamide, melphalan, chlorambucil, busulfan, nitrosurea, plicomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), α and γ interferon; docetaxel, actinomycin D (dactinomycin), mitomycin (also known as mutamycin and/or mitomycin-C), bleomycin, streptonigrin (SN, also known as rufochromomycin and bruneomycin), teniposide, paclitaxel, methotrexate, and vinorelbine; or any analog or derivative variant of the foregoing, but are not limited to such. The combination of chemotherapy with biological therapy is known as biochemotherapy.

2. Inhibitors of Cellular Proliferation

An adjunct therapy contemplated in the present invention includes inhibitors of cellular proliferation such as tumor suppressors oncogenes, but is not limited to such. Tumor suppressor oncogenes function to inhibit excessive cellular proliferation. The inactivation of these genes destroys their inhibitory activity, resulting in unregulated proliferation. The tumor suppressors p53, p16 and C-CAM are well known in the art. Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

In other embodiments of the invention an adjunct therapy contemplated is regulators of apoptosis or programmed cell death. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein is recognized to be a member of a family of related proteins, which can be categorized as death agonists or death antagonists.

Subsequent to its discovery, it was shown that Bcl-2 acts to suppress cell death triggered by a variety of stimuli. The Bcl-2 cell family of death regulatory proteins share common structural and sequence homologies. These different family members either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

4. Surgery

Approximately 60% of persons with cancer will undergo surgery of some type, which includes preventative, diagnostic or staging, curative and palliative surgery. Curative surgery is a cancer treatment that may be used in conjunction with other therapies, such as the therapeutic compositions of the present invention, chemotherapy, radiotherapy, hormonal therapy, gene therapy, protein therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying. dosages as well.

VI. Pharmaceutical Compositions and Delivery of Thioredoxin, Thioredoxin-like Molecules, Anticancer Compounds, Adjunct Therapies and Compositions Thereof

In particular embodiments of the present invention, a method for treating a neoplastic disease by the delivery of a thioredoxin or thioredoxin-like molecule molecule to a subject in combination with an anticancer therapy is contemplated. The anticancer therapy may be an anthracycline such as, but not limited to, doxorubicin or daunorubicin; or a radiotherapeutic agent. In other embodiments of the invention, the cancer may be treated by the delivery of a composition comprising a thioredoxin or thioredoxin-like molecule in combination with an anticancer agent, and an adjunct therapeutic agent to a subject. In other embodiments of the invention, a method of inhibiting, reducing, or eradicating a cancer, and/or promoting cell killing is also contemplated. Neoplastic diseases that are most likely to be treated with the compositions of the present invention include but are not limited to those of the lung, breast, head and neck, bladder, bone, bone marrow, brain, colon, esophagus, gastrointestine, gum, kidney, liver, nasopharynx, ovary, prostate, skin, stomach, testis, tongue, or uterus.

A. Delivery of Thioredoxin, Thioredoxin-like Molecules, Anticancer Therapies, Adjunct Therapies and Compositions Thereof

In some embodiments of the present invention, a method for treating a neoplastic disease by delivering a nucleic acid encoding a thioredoxin or thioredoxin-like molecule in combination with an anticancer therapy to a subject is contemplated. Such anticancer therapies may include but are not limited to anthracyclines or radiotherapeutics. In further embodiments of the invention, a nucleic acid encoding a composition comprising a thioredoxin or thioredoxin-like molecule in combination with an anticancer therapeutic agent and an adjunct therapeutic agent is contemplated for treating cancer. Virtually any method by which nucleic acids can be introduced into a cell or an organism may be employed with the current invention, as described herein or as would be known to one of ordinary skill in the art. Such methods include, but are not limited to direct delivery of DNA by: injection (U.S. Pat. Nos. 5,994,624, 5,981,274, 5,945,100, 5,780,448, 5,736,524, 5,702,932, 5,656,610, 5,589,466 and 5,580,859, each incorporated herein by reference); microinjection (Harland and Weintraub, 1985; U.S. Pat. No. 5,789,215, incorporated herein by reference); electroporation (U.S. Pat. No. 5,384,253, incorporated herein by reference; Tur-Kaspa et al., 1986; Potter et al., 1984); calcium phosphate precipitation (Graham and Van Der Eb, 1973; Chen and Okayama, 1987; Rippe et al., 1990); using DEAE-dextran followed by polyethylene glycol (Gopal, 1985); direct sonic loading (Fechheimer et al., 1987); liposome mediated transfection (Nicolau and Sene, 1982; Fraley et al., 1979; Nicolau et al., 1987; Wong et al., 1980; Kaneda et al, 1989; Kato et al., 1991) receptor-mediated transfection (Wu and Wu, 1987; Wu and Wu, 1988); microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); agitation with silicon carbide fibers (Kaeppler et al., 1990; U.S. Pat. Nos 5,302,523 and 5,464,765, each incorporated herein by reference); PEG-mediated transformation of protoplasts (Omirulleh et al., 1993; U.S. Pat. Nos. 4,684,611 and 4,952,500, each incorporated herein by reference); desiccation/inhibition-mediated DNA uptake (Potrykus et al., 1985); or any combination of such methods.

B. Injectable Compositions and Formulations

Compositions, compounds or agents of the present invention may be administered to a subject by any method known to one of ordinary skill in the art, for administering protein or gene therapy or a small molecule. Thus, compositions of thioredoxin or thioredoxin-like molecules, or anticancer therapies, or adjunct therapies; or the combination comprising thioredoxin or thioredoxin-like molecules with an anticancer therapy; or the composition comprising thioredoxin or a thioredoxin-like molecules with an anticancer therapy and an adjunct therapy, may be administered by any method known to those of skill in the art. These compositions may be preferably administered intravenously, intralesionally, precutaneously, subcutaneously or by inhalation to a subject. The thioredoxin or thioredoxin-like molecule or compositions thereof enhances the redox-cycling of an anticancer therapeutic agent thereby increasing apoptosis. In some embodiments, the pharmaceutical compositions disclosed herein may alternatively be administered parenterally, intrapleurally, intradermally, intramuscularly, transdermally or even intraperitoneally as described in U.S. Pat. No. 5,543,158; U.S. Pat. No. 5,641,515 and U.S. Pat. No. 5,399,363.

Injection of a thioredoxin or thioredoxin-like molecule and its related compounds in combination with an anticancer agent or compositions comprising a thioredoxin or thioredoxin-like molecule in combination with an anticancer agent and an adjunct agent may be administered by syringe or any other method used for injection of a solution, as long as the molecules can pass through the particular gauge of needle required for injection. A novel needleless injection system has been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

C. Dosage and Schedules of Administration

The dosage of the active compound and dosage schedule may be varied on a subject by subject basis, taking into account, for example, factors such as the weight and age of the subject, the type of disease being treated, the severity of the disease condition, previous or concurrent therapeutic interventions, the manner of administration and the like, which can be readily determined by one of ordinary skill in the art.

Administration is in any manner compatible with the dosage formulation, and in such amount as will be therapeutically effective. The quantity to be administered depends on the subject to be treated, including, e.g., the capacity of the individual's immune system to synthesize antibodies, and the degree of protection desired. The dosage will depend on the route of administration and will vary according to the size of the host. Precise amounts of an active ingredient required to be administered depend on the judgment of the practitioner.

In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. However, a suitable dosage range may be, for example, of the order of several hundred micrograms active ingredient per vaccination. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per vaccination, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. A suitable regime for initial administration and booster administrations (e.g., inoculations) are also variable, but are typified by an initial administration followed by subsequent inoculation(s) or other administration(s).

In many instances, it will be desirable to have multiple administrations, usually not exceeding six administrations, more usually not exceeding four administrations and preferably one or more, usually at least about three administrations. Normally from two to twelve week intervals, more usually from three to five week intervals may be applicable.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Experimental Procedure Using Cancer Cells Expressing Thioredoxin

Cell culture and adenovirus production. MCF-7 cells were cultured in DMEM with 10% fetal bovine serum and 100 units of penicillin/streptomycin. MCF-7 clones expressing thioredoxin (Trx9) or only vector (Vector) have been described (Oblong et al., 1994). MCF-7 clones were cultured in DMEM containing G418 (300 jig/ml), gentamycin (100 μg/ml) and ampicillin (100 μg/ml). AdenoX system was obtained from Stratagene Corporation (La Jolla, Calif.) and thioredoxin or mutant thioredoxin ORF (Das, 2001) was cloned into the Not I site of the pAdenoX vector. Recombinant virus was allowed to infect HEK293 cells for generation of viral particles.

TUNEL assay. Apoptotic cells were detected using In Situ Cell Death detection, POD kit (Roche Molecular Biochemicals, Indianapolis, Ind.). Apoptotic DNA strand breaks were identified by labeling 3′-OH termini with fluorescein-dUTP using Terminal deoxy Transferase as per manufacturer's protocol. For this assay cells were allowed to adhere overnight in chambered glass slides (Nunc) to a final density of 25,000 cells per well. Followed by treatment with the appropriate concentration of drugs the media was removed and washed twice with PBS containing 1% BSA and the cells fixed in 4% paraformaldehyde for 30 min and washed with PBS containing 1% BSA. The cells were then permeabilized with 0.1% Triton X-100 in 0.1% sodium citrate for 2 min on ice and washed twice with PBS containing 1% BSA. The labeling reaction was performed using FITC labeled dUTP along with other nucleotides by terminal deoxynucleotidyl transferase for 60 mins in dark at 37° C. in humidified chamber. The cells were washed with PBS-1% BSA, mounted onto a slide, and the incorporated fluorescein-dUTP was analyzed using Nikon laser confocal microscope.

Flow cytometry. Cells were treated with drugs for 48 hr, floating cells were collected, and adherent cells were washed with phosphate buffered saline (PBS), and trypsinized and pooled with floating cells and centrifuged at 500×g for 3 min. The cells were washed again with PBS containing 1% FBS and resuspended in 500 μl of PBS followed by fixing in 7.5 ml of ice-cold ethanol 70% added dropwise while vortexing and stored in −20° C. overnight. After two washes with PBS containing 1% FBS and resuspended in same buffer and stained with Propidium Iodide 10 μg/ml (Sigma, St. Louis, Mo.) in the presence of RNase 250 μg/ml at 37° C. for 30 min in dark. Stained cells were analyzed using Epics Elite ESP Coulter, using argon laser at 488 nm wavelength. Flow cytometric results were analyzed and apoptosis was defined as ‘sub G1’ peak (Gutierrez, 2000) using Multicycle software.

Cell proliferation assay. Cell proliferation assay by BrdU ELISA kit was purchased from Roche Molecular Biochemicals (Indianapolis, Ind.). For this assay cells were seeded to a final density of 5000 per well in 96 well plates and allowed to attach overnight, followed by treatment with drugs for 48 hr followed by labeling with BrdU for 4-6 hr, and detected using Tetramethylbenzedine as substrate with Anti-BrdU POD at 370 nm and reference wavelength 492 nm in Spectramax 190 plate reader.

XTT assay. Cytotoxicity was estimated using XTT based in vitro toxicology assay kit, (Sigma Chemical Co.). This assay is based on the metabolic capacity of viable cells; the mitochondrial dehydrogenases convert XTT yielding orange formazan crystal soluble in aqueous media that is estimated spectrophotmetrically at 450 nm and a reference wavelength at 690. The assay was performed 48 hr post treatment; cells were washed and XTT added in media free of serum and phenol red, incubated for 2-4 hr and analyzed in Spectramax 190 plate reader.

Preparation of cytosolic extract by sub cellular fractionation. Cytosolic preparation was done as described earlier (Yang et al., 1997). Briefly, cells were harvested by centrifugation (600×g) and washed once with ice cold PBS for 10 min at 4° C. The cell pellets were resuspended in five volumes of fractionation buffer (250 mM sucrose containing 20 mM HEPES-KOH, 10 mM KCl, 1.5 mM each of sodium-EDTA and sodium- EGTA, 1 mM MgCl₂, 1 mM dithiothreitol, 0.1 mM PMSF and cocktail of protease inhibitors) and incubated on ice for 30 min. Cells were then homogenized using a dounce homogenizer with type B pestle and centrifuged twice at 800×g for 10 min at 4° C. The supernatant was centrifuged at 10,000×g for 15 min at 4° C., and the resulting supernatant was then transferred to fresh tube and centrifuged at 100,000×g 8 for 1 hr at 4° C. The final supernatant obtained in this centrifugation was designated as cytosolic extract and was stored in −70° C. for further analysis.

Western blotting. Equal amounts of protein were resolved on 10% SDS-polyacrylamide gel electrophoresis, and transferred to nitrocellulose membrane (Hybond-ECL, Amersham Pharmacia Biotech) immunoblotted with anti-p53 or anti-p53 phospho ser-15 (Cell Signaling Technology, Beverly, Mass.) and visualized using Lumiglo system with antirabbit conjugated HRP. For cytochrome c determination the cytosolic protein was resolved on 15% SDS-PAGE and immunoblotted using anti-cytochrome c (BD Pharmingen, San Diego, Calif.) and visualized using the ECL system (Amersham Pharmacia Biotech, Piscataway, N.J.) and anti-mouse IgG conjugated HRP (Santa Cruz Biotech, California).

Colony formation assay. For determination of clonogenic survival, the cells were seeded to a final density of 1×10⁶ in 100 mm dishes, allowed to attach overnight, and the following day treated with the appropriate concentration of the drug for 48 hr. The cells were trypsinized, counted and seeded to final density of 25,000 viable cells in 100 mm dishes and allowed to grow for 14 days followed by washing and staining with crystal violet.

Determination of superoxide production by reduction of ferricytochrome c. Superoxide production was measured as superoxide dismutase (SOD) inhibitable reduction of ferricytochrome c (Azzi et al. 1975). Cells were sonicated in potassium phosphate buffer (0.05M, pH 7.8 plus 1 mM EDTA), centrifuged and the supernatant was used for the assay. To determine the superoxide generation in the cell lysate, the supernatant was incubated with 10 μM drug and 10 μM cytochrome c, with or without 1U SOD to determine SOD inhibitable rate. The specificity of thioredoxin mediated superoxide generation in the supernatant was detected by pre-incubation of cell lysates with anti-human thioredoxin antibody (American Diagnostica, Greenwich, Conn.) for 15 min. All reactions were performed in triplicate. The reduction of ferricytochrome c was measured both in kinetic and end point mode with path check on for 1 hr duration at a wavelength of 550 nM using a Spectramax 190 plate reader (Molecular Devices). Total protein was quantified using Bradford protein assay (Biorad) and the superoxide generation was determined per milligram of protein in the extracts.

In situ detection of superoxide by fluorescent probe DiCarboxyFluorescein-DiAcetate (DCF-DA). Cells were grown in chambered glass slides (Nunc) to a final density of 25,000 cells per well. Cells were pre-incubated with 20 μM DCF-DA (Sigma, St. Louis, Mo.) in 20 mM Hepes in Phosphate Buffered Saline (PBS) containing bovine serum albumin (BSA, 5 mg/ml) at 37° C. for 30 min followed by washing with PBS buffer, and the drug was added and observed for 10-15 min in a Nikon laser confocal microscope using laser beam wavelength 488 nm analyzed by Ultraview software (Perkin-Elmer).

Nuclear Extract preparation. Nuclear extract was prepared as described previously (Das, 2001). Briefly, cells were washed in ice cold PBS and harvested in 2 ml of ice cold PBS by centrifugation, followed by removal of PBS the cell pellets were resuspended in 400 μl of Buffer A (10 mM HEPES, pH 7.8, 10 mM KCl, 0.1 mM EDTA, 1 mM Dithiothreitol, 1 mM Phenylmethylsulfonyl fluoride (PMSF) and 50 μg/ml of leupeptin and antipain by gentle pipetting. The cells were allowed to swell on ice for 15 min followed by addition of 25 μl 10% Nonidet-P40 and vortexed at full speed for 10 sec. The homogenate was centrifuged 30 s at 14,000 rpm. The nuclear pellet was resuspended in buffer C (20 mM HEPES, pH 7.8, 0.42 M NaCl, 5 mM EDTA, 1 mM Dithiothreitol, 1 mM PMSF in 10% v/v glycerol) and the tubes rocked gently at 4° C. for 30 min on a shaking platform. The extracts were then centrifuged at 14,000 rpm for 25 min and the supernatant was saved as nuclear extract in −70° C. for further experiments. Protein was quantified using Bradford protein assay (Biorad).

Electrophoretic Mobility shift assay (EMSA). For the EMSA the p53 consensus oligonucleotide was obtained from Genosys 5′-GGCATGTCCGGGCATGTCC-3′ (SEQ ID NO:9) and end labeled using T4 Poly nucleotide kinase (New England Biolabs, Beverly, Mass.) and [γ-³²P] ATP (Perkin Elmer, Boston, Mass.) in 10× kinase buffer supplied with the enzyme. Ten microgram of nuclear protein was pre-incubated in 51 l of 5× binding buffer (20% glycerol, 5 mM MgCl₂, 5 mM EDTA, 5 mM DTT, 500 mM NaCl, 50 mM Tris HCL, 0.4 mg/ml calf thymus DNA), 200 ng anti-p53 pAb 421 and 2 μg of poly dIdC for 15 min followed by binding with labeled oligonucleotide for 30 min. The nuclear protein was separated by electrophoresis using 4% native polyacrylamide gel and 0.25× of TBE (Tris-Borate-EDTA) as running buffer. The gels were dried and exposed to Kodax Biomax X-ray film overnight.

### Example 2

### Results Using Cancer Cells Expressing Thioredoxin

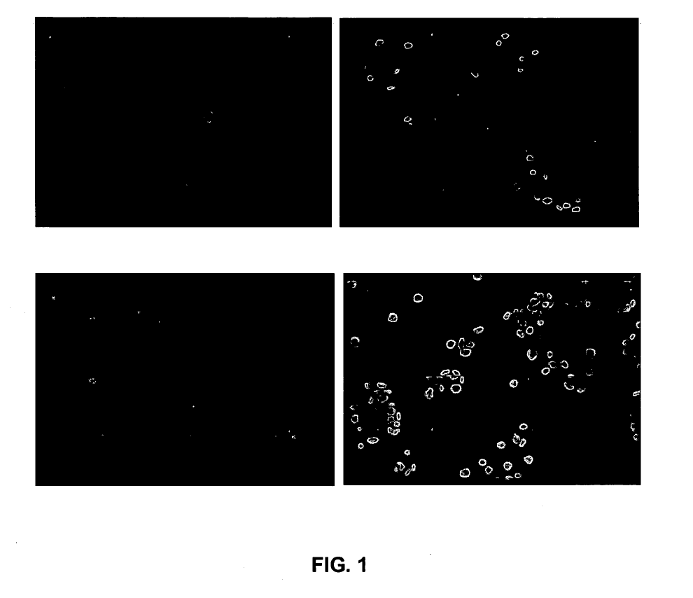

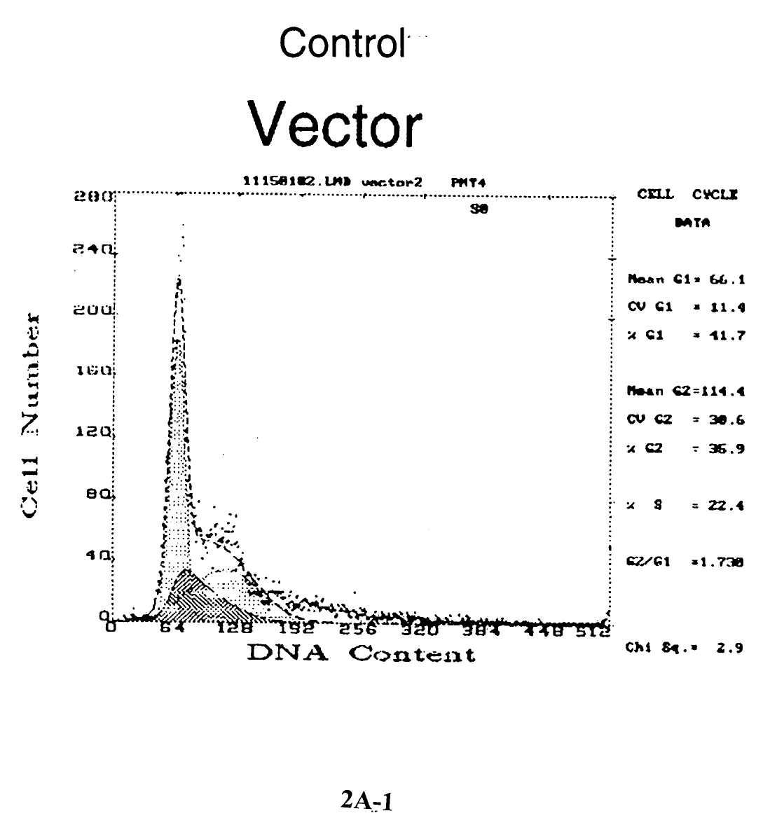

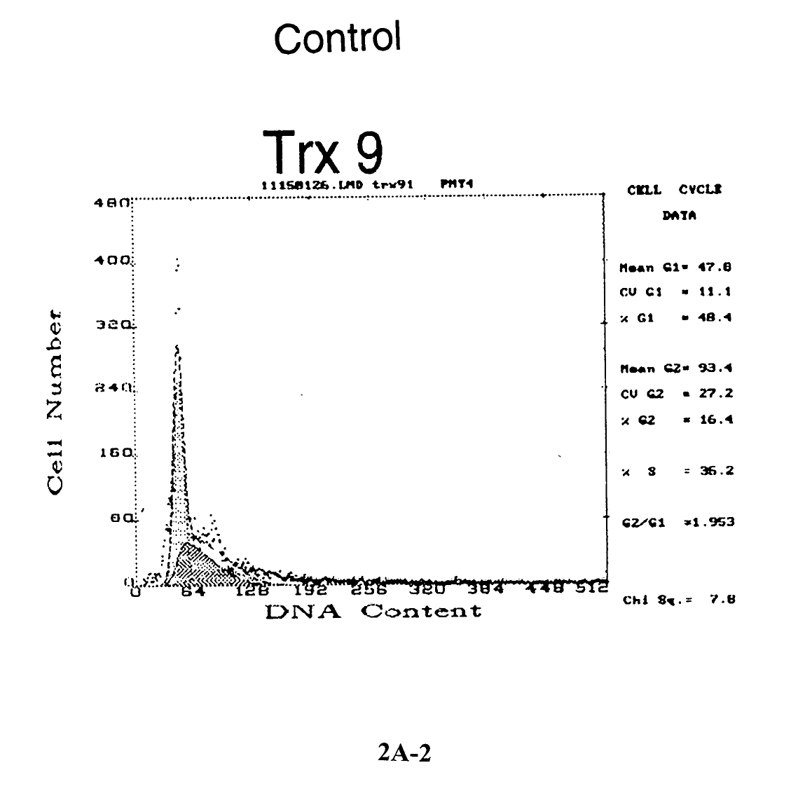

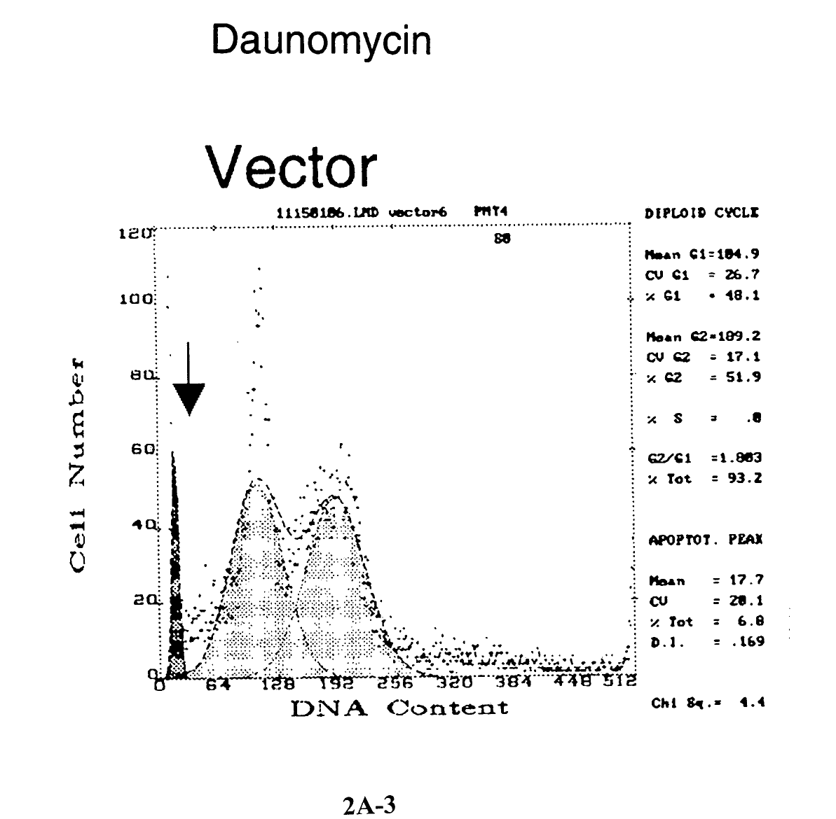

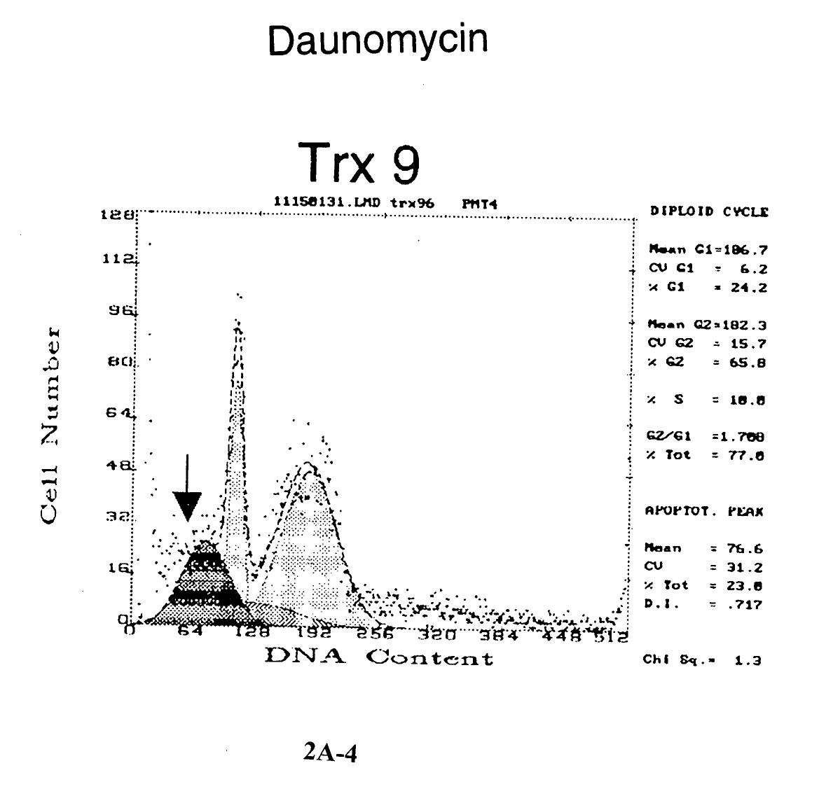

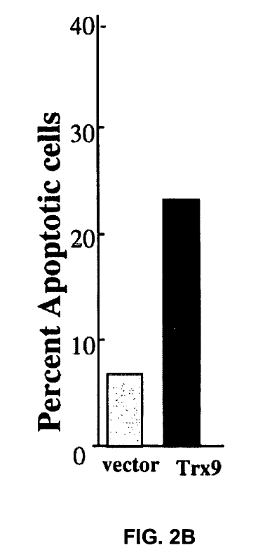

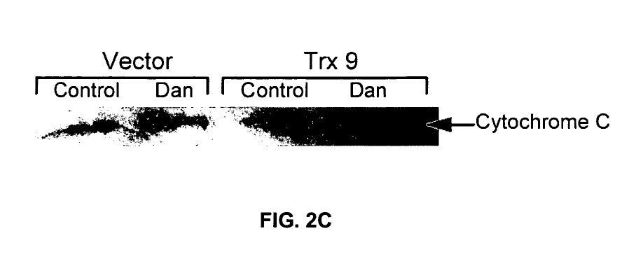

Increased apoptosis in thioredoxin-over expressed MCF-7 (Trx9) cells in response to anticancer drugs. To determine whether thioredoxin overexpression protects MCF-7 cells against anthracycline mediated apoptosis, vector-only MCF-7 cells or Trx9 cells were treated with daunomycin as described in the experimental procedures and TUNEL-positive cells were detected. A higher number of TUNEL positive Trx9 cells were observed as compared to TUNEL-positive vector cells (FIG. 1), which indicated that MCF-7 cells undergo increased apoptosis in the presence of thioredoxin (FIG. 1) in response to anthracyclines. To further determine the role of thioredoxin in apoptosis mediated by anthracyclines, the cell cycle changes were analyzed and apoptosis detected, as ‘sub G1 peak’, by propidium iodide staining. Increased expression of thioredoxin in MCF-7 cells (Trx9) demonstrated pronounced changes in the cell cycle distribution (FIG. 2A). These changes include increased S-phase population in Trx9 cells (35%) compared to vector (22%); decreased G2-phase in Trx9 cells (16%) compared to vector (36%). Rapid entry in to S-phase and shorter G2 in Trx9 cells demonstrated that Trx9 cells have higher proliferative potential when compared to vector only cells. Treatment of cells with daunomycin or doxorubicin resulted in the appearance of apoptotic cells (sub-G1), and alteration of distribution of cell cycle phases (FIG. 2A and FIG. 2B). Thus, Trx9 cells showed a larger percentage of apoptotic cells (23%) compared to vector (6%, FIG. 2B).

Release of cytochrome c in Trx9 cells in response to daunomycin treatment. Translocation of cytochrome c from mitochondria to the cytosol has been shown to be a crucial step in the activation of apoptosis (Yang et al., 1997). Once released, cytochrome c in interaction with at least one other cytoplasmic component, initiates the activation of execution caspases, which leads to subsequent characteristic features of apoptosis, including chromatin condensation and nuclear fragmentation (Yang et al., 1997). Therefore, release of cytochrome c has been used as a marker of apoptosis. As demonstrated in FIG. 2C, Trx9 cells treated with daunomycin demonstrated increased cytochrome c release compared to vector-only cells. These data further confirm that Trx9 cells treated with daunomycin are more apoptotic compared to vector cells.

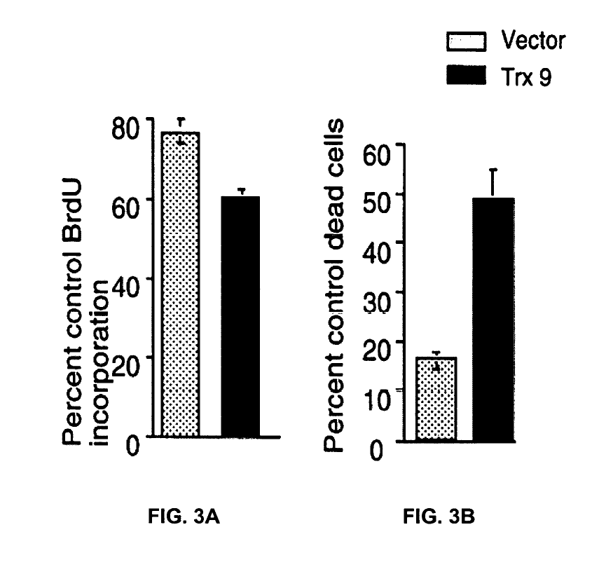

Anthracyclines are more cytotoxic to Trx9 cells. To determine the effect of thioredoxin overexpression on the cytotoxicity of anthracyclines, BrdU incorporation into vector or Trx9 cells was determined following treatment with drugs. As shown in FIG. 3A, Trx9 cells incorporated lesser level of BrdU (60%) compared to vector cells (76%). Additionally, as shown in FIG. 3B there was higher percentage of cell death in Trx9 cells (48%) compared to vector cells (16%). Thus, there was significant death in Trx9 cells. These data demonstrated that Trx9 cells are more cytotoxic to anthracyclines compared to vector cells.

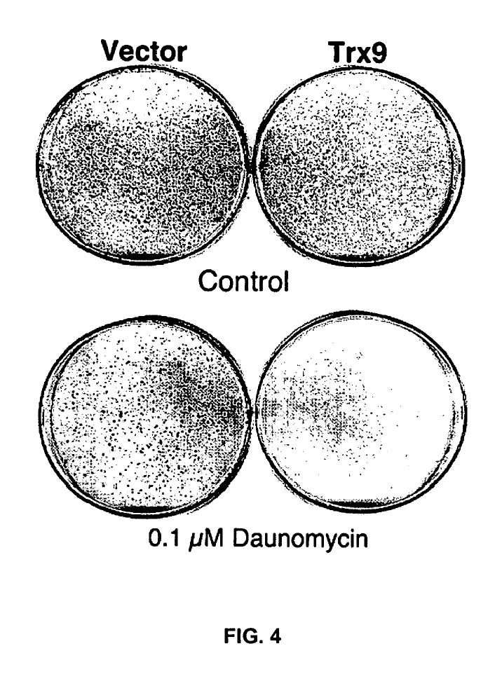

Decreased clonogenic survival of Trx9 cells in response to daunomycin. In response to cytotoxic treatment, cells can survive DNA damage through various repair processes, and can continue to propagate into colonies, depending on the extent or severity of drug-induced damage (Hwang et al., 2001). Therefore, clonogenic survival in response to anthracyclines was determined by comparing the sensitivity of Trx9 and vector cells in response to treatment with doses as low as 0.1 μM daunomycin. A clonogenic assay is more stringent than TUNEL or Sub-G1 peak measurements in determining the apoptotic death of cells (Hwang et al., 2001). As shown in FIG. 4, Trx9 cells treated with daunomycin failed to propagate into colonies at the end of 14 days, whereas vector cells treated with daunomycin were able to propagate into several colonies. These results show that MCF-7 cells overexpressing thioredoxin exhibit increased apoptosis and decreased clonogenic survival, as compared to vector-transfected cells.

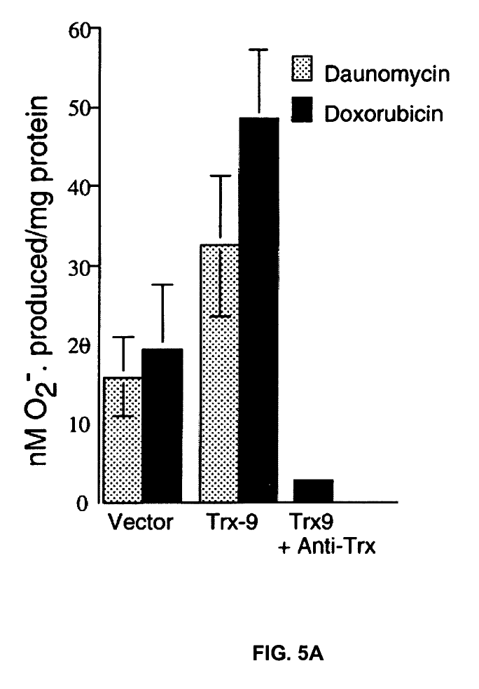

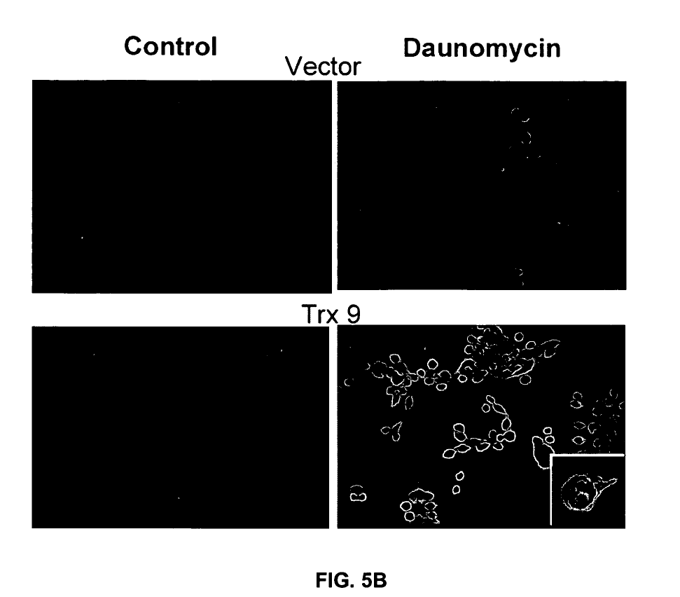

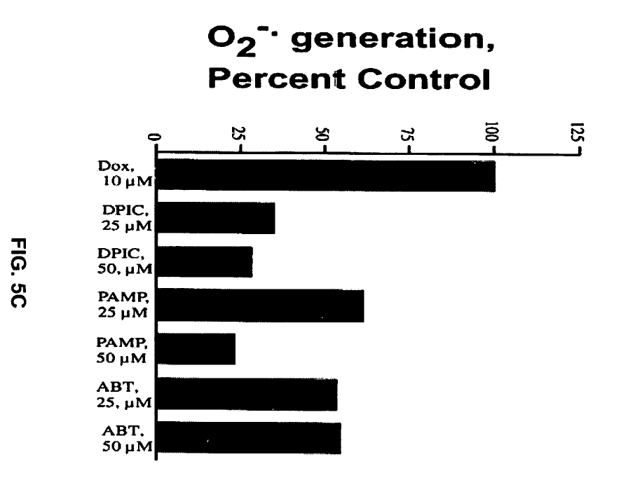

Anthracycline treatment increases the generation of superoxide anions in Trx9 cells. Cytotoxicity and apoptosis assays demonstrated that Trx9 cells undergo increased cell death and apoptosis in response to daunomycin as compared to vector-transfected control cells. Because anthracycline antibiotics undergo redox-cycling and generate O₂—, it was reasoned that daunomycin may increase O₂— production in Trx9 cells. Additionally, generation of O₂— is also a measure of redox-cycling (Gutierrez, 2000). This was investigated by measuring the production of O₂— in the presence of anthracyclines in Trx9 or vector cells. As shown in FIG. 5A, both doxorubicin and daunomycin produced significantly higher level of O₂— in Trx9 cells, as compared to vector-transfected cells. The specificity of this reaction was assessed by pre-incubating cell extracts with an anti-thioredoxin antibody (American Diagnostica, Conn.). Incubation with anti- thioredoxin antibody blocked the generation of O₂— in the presence of drugs, suggesting that thioredoxin is essential for redox-cycling of the anthracyclines. Next, the total load of oxidative stress was estimated by using the ROS sensitive probe 2′,7′-dichloroflourescin diacetate (DCF-DA) to measure the total cellular peroxide levels in vector or Trx9 cells in response to daunomycin. Pre-incubation of cells with non-fluorescent DCF-DA dye followed by treatment with doxorubicin resulted in enhanced fluorescence due to increased oxidation of DCF-DA in Trx9 cells compared to vector cells (FIG. 5B). Thus, different methods confirm that Trx9 cells produce more O₂— and higher level of ROS than vector cells in response to anthracyclines.

Effect of bioreductive enzyme inhibitors on superoxide anion generation in Trx9 cells. It was observed that Trx9 cells generate more O₂— in the presence of anthracyclines. Redox-cycling of anthracyclines has been shown to be mediated by one electron reduction by NADPH-cytochrome P-450 reductase (Komiyama et al., 1985), mitochondrial NADH dehydrogenase (Pan et al., 1981) and nitrate reductase (Pan and Bachum, 1980) from Neurospora. Therefore, to determine the involvement of bioreductive enzyme(s) in the redox-cycling of daunomycin in the presence of increased thioredoxin, a variety of pharmacologic inhibitors of several bioreductive enzymes were used. These included 1-Aminobenzotriazole for inhibition of cyt-p450 reductase (ABT, Mathews et al., 1985), dipheyleneiodonium (DPIC, Osada et al., 2002) as a general inhibitor of NADPH-dependent reductases, and 2′-Phospho-5′-AMP for inhibition of ferredoxin reductase (PAMP, Bruns and Karplus, 1995), and as demonstrated in the FIG. 5C, ferredoxin reductase inhibitor PAMP, and Cyt-P450-reductase inhibitor ABT 16 which inhibited the generation of SOD-inhibitable rate of O₂— by 76% and 54% respectively at 50 μM concentrations. The generation of O₂— in this treatment was considered 100%. These results indicated the involvement of both of these reductases in the redox-cycling of anthracyclines using reducing equivalents from reduced thioredoxin. Additionally, DPIC a non-specific reductase inhibitor also inhibited generation of O₂— indicating that thioredoxin provides electrons to bioreductive enzymes for anthracyline redox-cycling.

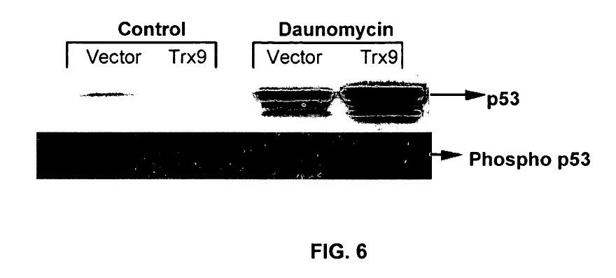

Anthracyclines increase p53 protein level in Trx9-cells. Anticancer agents that induce DNA damage also induce p53-mediated apoptosis (Sadji-Ouatas et al., 2002; Maya et al., 2001). In the event of DNA damage, p53 is activated by phosphorylation and then binds to the DNA, inducing several effector genes. This also stabilizes the p53 protein, thereby increasing p53 level. In response to DNA damage, cells undergo a sequence of events that result either in growth arrest and repair, or apoptosis (Maya et al., 2001). The findings show that ROS generation is enhanced in Trx9 cells in the presence of anthracyclines. This suggested that treatment with daunomycin may cause extensive DNA damage, thereby leading to p53-mediated cell death in Trx9 cells. In order to determine the role of thioredoxin in enhancing the p53-dependent cytototoxicity of daunomycin, the level and the extent of phosphorylation of p53 in Trx9 cells was examined. Treatment with 1 μM daunomycin increased p53 protein in both vector and Trx9 cells (FIG. 6, upper panel), although basal p53 level was 2-fold higher in Trx9 cells than in vector-transfected cells. This finding suggested that Trx9 cells were much more sensitive to the damaging effects of daunomycin. Next, the phosphorylation state of p53 in daunomycin treated cells was evaluated by using phospho-specific antibodies (Ser-15). Trx9 cells exhibited higher phospho-p53 (ser-15) level in response to daunomycin, as compared to vector cells, suggesting that p53 is highly activated in Trx9 cells (FIG. 6, lower panel).

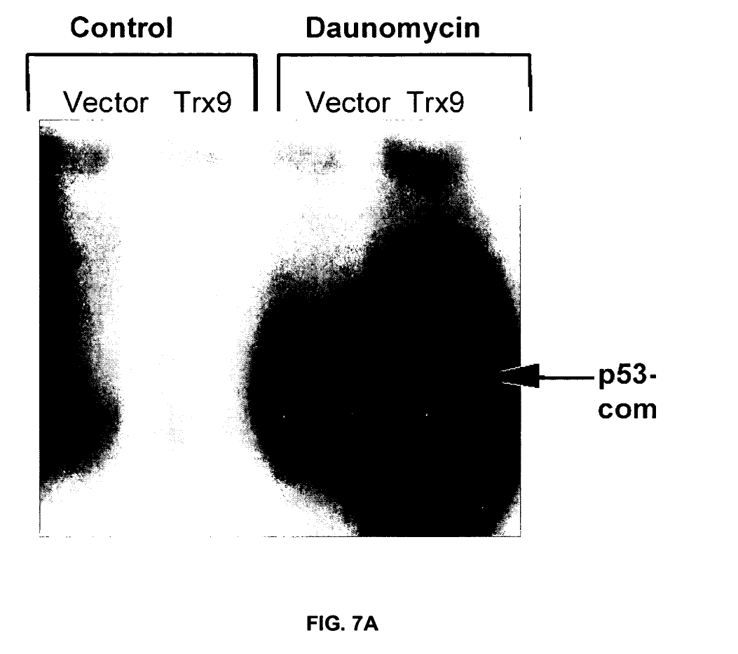

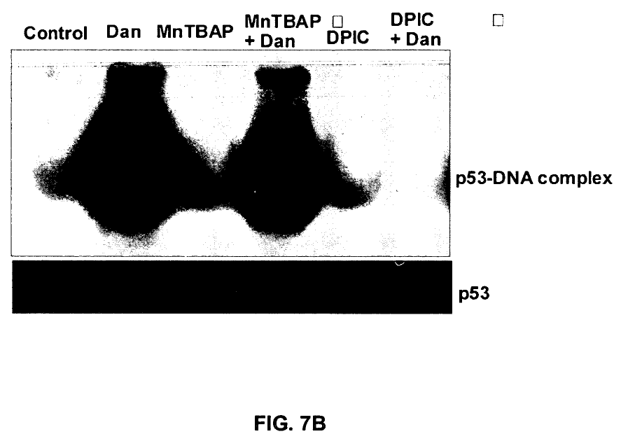

Anthracyclines increase p53 DNA binding in Trx9 cells. In response to DNA damage, p53 acts as a nuclear transcription factor that regulates the expression of many different genes involved in apoptosis or DNA repair by its ability to bind to consensus sequences in the promoters of various genes. Thus, electrophoretic mobility shift assays (EMSA) was used to measure p53 binding to the DNA in nuclear extracts of cells treated with or without daunomycin. As demonstrated in FIG. 7A, daunomycin-induced p53 DNA binding were several folds higher in Trx9 cells than in vector cells. Increased DNA binding suggested increase activation of the p53 protein, an thus an increase in apoptosis. These results are also consistent with the increased superoxide production exhibited in Trx9 cells compared to vector cells, suggesting that thioredoxin cells may be undergoing extensive damage. Such damage is expected to recruit more p53 protein and trigger apoptosis.

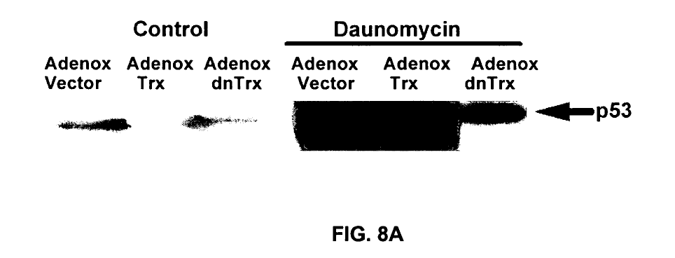

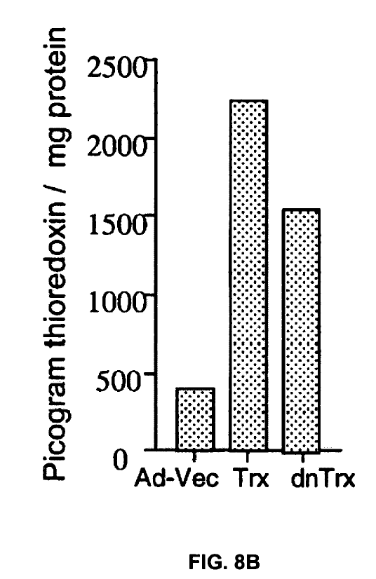

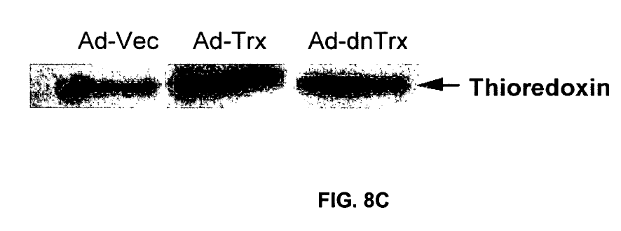

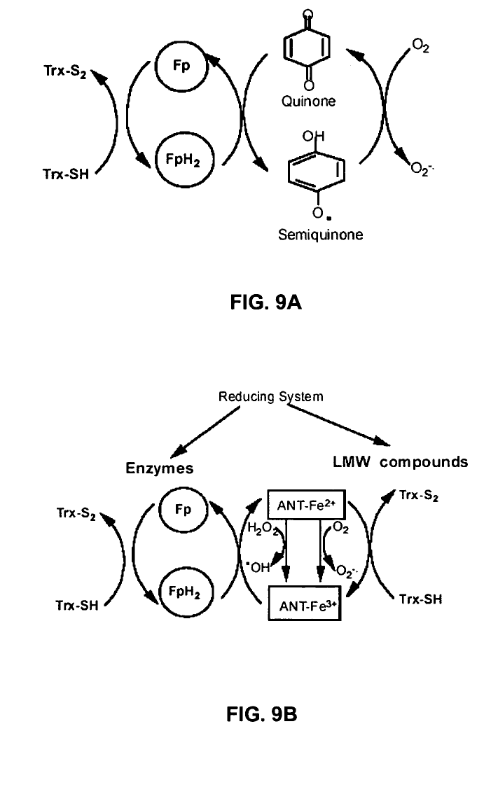

Effect of O₂— scavenger MnTBAP and reductase inhibitor DPIC on p53 binding and ser-15 phosphorylation in Trx9 cells. To understand the relative contribution of O₂— and semiquinone radical due to redoxcycling on p53 activation, MnTBAP was used to scavenge O₂— (Osada et al., 2002) in daunomycin treated Trx9 cells. As demonstrated in FIG. 7B (upper panel), MnTBAP treatment resulted in about 30% (densitometric analysis) reduction in the binding activity. This data indicated that activation of p53 is not fully dependent on O₂—. Ser-15 phosphorylation in response to MnTBAP treatment was also determined. As demonstrated in FIG. 7B (lower panel), there was no appreciable decrease in ser-15 phosphorylation in response to MnTBAP. These data further suggested that p53 continued to be activated in the absence of O₂—. Next, the role of reductases that redox-cycle the anthracyclines was determined. Treatment of cells with a general reductase inhibitor DPIC abolished p53 DNA binding as well as p53 (ser-15) phosphorylation (FIG. 7B). These data indicated that generation of semiquinone by redox-cycling may contribute to p53 activation as a result of DNA damage in Trx9 cells. Transient overexpression of thioredoxin increased p53 protein level in response to daunomycin. It was demonstrated that MCF-7 clones (Trx9) stably expressing higher level of thioredoxin showed increased p53 protein expression in response to daunomycin compared to vector only cells. Individual clones may demonstrate unique characteristics that may be different than the parental MCF-7 cells. Therefore, the level of thioredoxin was transiently increased using adenovirus-mediated gene delivery as described in the experimental procedures, and its effect on daunomycin-induced p53 expression was investigated. Overexpression of thioredoxin and mutant thioredoxin protein was determined using ELISA as described earlier (Das and White, 1998; FIG. 8B) and also by western blotting (FIG. 8C). As demonstrated in FIG. 8A, daunomycin treatment increased p53 protein level in MCF-7 cells transiently overexpressing thioredoxin. In contrast, there was little or no p53 protein increase in MCF-7 cells transiently overexpressing redox-inactive mutant thioredoxin protein. These results suggested that in the absence of redox-active thioredoxin there could be less redox- cycling of anthracyclines resulting in lower p53 induction.

### Example 3

### Experimental Procedures Using Cells Expressing E. coli Thioredoxin

Reagents. E. coli thioredoxin was obtained from Promega Inc, USA. E. coli thioredoxin reductase was obtained from American Diagnostica Inc, Greenwich, Conn. Cytochrome c (partially acetylated), daunomycin, doxorubicin, NADPH, NADPH cytochrome P450 reductase (Rabbit liver) and superoxide dismutase were purchased from Sigma Chemicals (St.Louis. Mo.).

Superoxide detection by cytochrome c reduction assay. Superoxide (O₂—) generated in the process of redox-cycling was detected by SOD inhibitable rate of ferricytochrome c reduction (Das et al., 2000). The reaction mixture contained 0.05 M potassium phosphate buffer (pH 7.78), 1 mM EDTA, 10 μM cytochrome c and 200 μM NADPH. For detection of SOD inhibitable rate, 1-5 units of SOD was included in the assay; daunomycin or doxorubicin was used in a final concentration of 10 μM. The rate of oxidation of cytochrome c was measured at 550 nm for 0-15 minutes using Shimadzu UV-1601PC in kinetic mode. Reaction blank contained potassium phosphate buffer, cytochrome c and NADPH with or without SOD, and controls included 0.35-0.7 μg TR and /or Trx in buffer. All reactions were performed in triplicate. Cytochrome-P450 reductase mediated SOD inhibitable rate of superoxide generation was determined using reduced thioredoxin as an electron donor to redox cycle doxorubicin in the similar method as described above.

Preparation of reduced Thioredoxin. Reduced thioredoxin was prepared by incubating 850 μM E.coli thioredoxin with 2 mM dithiothreitol (DTT) for 30 mins at 37° C. Excess DTT was removed using Biospin-6 desalting columns (Biorad) equilibrated with 10 mM Tris pH 7.0. The concentration of reduced thioredoxin was determined using molar extinction coefficient of 13,700 at A₂₈₀.

Cytochrome P450 reductase activity assay. Cytochrome P450 reductase activity was determined based on the method described (Das et al., 1992) with modifications. The reaction mixture contained 0.05 M potassium phosphate buffer (pH 7.7), 0.04U of purified rabbit liver cytochrome P450 reductase enzyme, and 50 μM cytochrome c. The reaction was initiated by addition of 11.2 μM of Trx-SH or Trx-S₂ and assayed for 0-5 minutes at 550 nm in kinetic mode (Shimadzu UV-1601PC).

Plasmid DNA damage assay. One microgram of plasmid pGL3 was incubated with 10 μM doxorubicin or 10 μM daunomycin in potassium phosphate buffer (0.05 M, pH 7.8) consisting of thioredoxin reductase-thioredoxin enzymes and NADPH with or without SOD. The control plasmid was incubated with drugs with NADPH but without thioredoxin reductase-thioredoxin enzymes. The reaction mixture was lyophilized, resuspended in 1X DNA gel loading buffer (6X gel loading buffer: 0.25% each bromophenol blue and xylene cyanol FF and 30% glycerol in water), and analyzed by 1% agarose gel electrophoresis, visualized with ethidium bromide staining, and photographed using Biorad gel documentation system.

Carboxymethylation of thioredoxin. In order to analyze the possible role of thioredoxin in the redox-cycling of anthracyclines the redox status of thioredoxin was determined by carboxymethylation at the end of assay. Carboxymethylation was done as described previously (Das et al., 1997). Equal volume of carboxymethylation buffer (0.1 M Tris; pH 8.8, 12 mg/ml iodoacetic acid, 3 mM EDTA, 7 M guanidine hydrochloride, and 0.5% Triton X-100, equilibrated with nitrogen for 1 h) was added and incubated at 37° C. in the dark for 45 min. After incubation the excess reagent was removed using Sephadex G-25 spin column, and the elute was used for western blot detection of thioredoxin.

Western analysis of thioredoxin redox state. Equal amount of protein was fractionated along with standards of E.coli reduced thioredoxin and oxidized thioredoxin on a 15% native polyacrylamide gel. The protein was transferred to PVDF membrane using a miniprotein transblot apparatus (Bio-Rad). The PVDF membrane was washed and incubated with mouse anti-E. coli Trx IgG (MBL, Nagoya, Japan). After washing, the blot was incubated with anti Mouse IgG- horseradish peroxidase conjugate for 1 h at room temperature. Binding of secondary antibody was detected using an enhanced chemiluminescence (ECL Plus) detection system (Amersham Pharmacia Biotech, Piscataway, N.J.). Western blot was quantified using densitometric analysis software (NIH Image 1.61).

TUNEL Assay and In situ detection of reactive oxygen species (ROS) by fluorescent probe DiCarboxyFluorescein-DiAcetate (DCF-DA). For these assays A549 cells were allowed to adhere overnight in chambered glass slides (Nunc) to a final density of 25,000 cells per well. The assays were conducted as described previously above for the MCF (trx-9) cells.

### Example 4

### Results Using Cells Expressing E. coli Thioredoxin

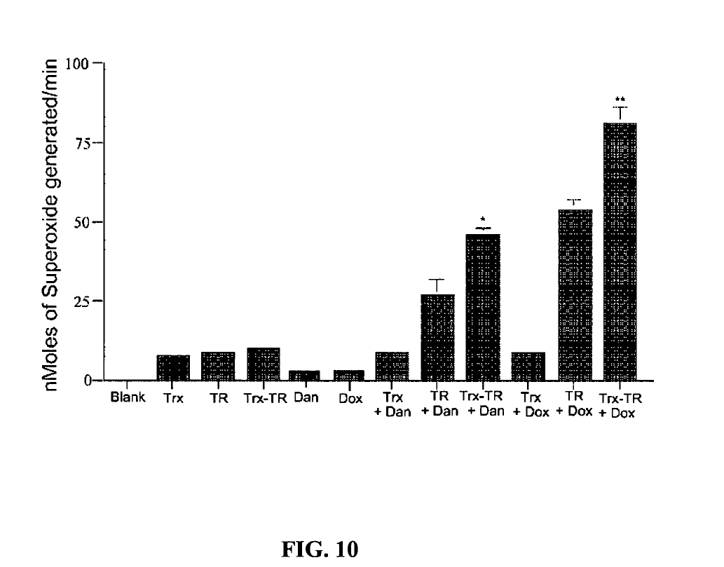

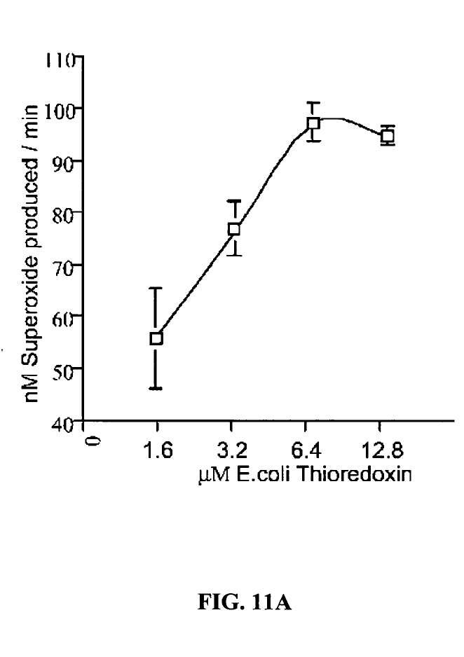

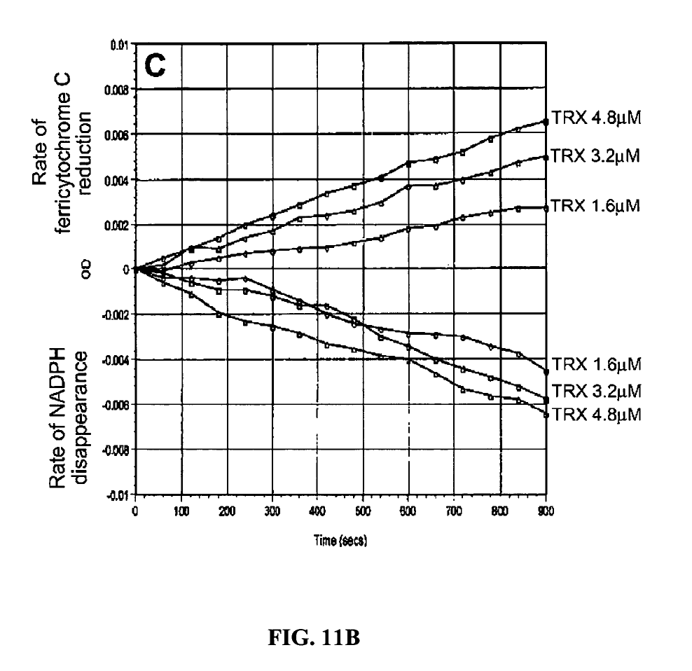

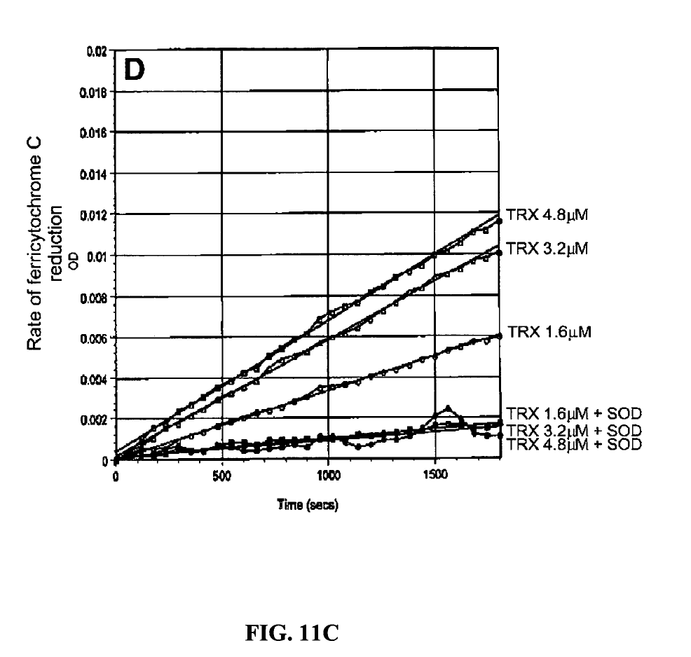

Anthracyclines generate superoxide anion with thioredoxin system. Addition of daunomycin or doxorubicin to thioredoxin system enhanced O₂— generation as detected by SOD inhibitable rate of reduction of ferricytochrome c. Thioredoxin or thioredoxin reductase individually or in combination did not induce significant level of SOD-inhibitable rate of ferricytochrome c reduction (FIG. 10). Doxorubicin or daunomycin alone also did not show significant level of SOD inhibitable rate of ferricytochrome c reduction (FIG. 10). Thioredoxin reductase alone generated significantly higher levels of O₂— with daunomycin or doxorubicin. Addition of 3.2 μM Trx further enhanced the levels of O₂— generated in these reactions (FIG. 10). Thioredoxin reductase alone or thioredoxin reductase-thioredoxin system showed two fold increase in the rate of superoxide generation in the presence of doxorubicin as compared to daunomycin (FIG. 10). By increasing the concentration of thioredoxin from 1.6 to 6.4 μM in reactions with 0.35 μg thioredoxin reductase, proportional increase in the rate of O₂— generation with doxorubicin was observed (FIGS. 11A-C). generation with doxorubicin (FIG. 11B). Further increases in Trx concentration did not increase the rate of 0₂ generation, indicating that a saturation concentration of Trx had been reached (FIG. 11B). Reactions were modified to adapt simultaneous determination of the rates of reduction of ferricytochrome c and the disappearance of NADPH in a microtiter assay containing 1.6-4.8 lM Trx with 0.35 lg/ml TR and 10 lM doxorubicin. A proportional increase in the rate of ferricytochrome c reduction and disappearance of NADPH was observed (FIG. 11C). The addition of SOD to these reactions inhibited the rate of ferricytochrome c reduction (FIG. 11D).

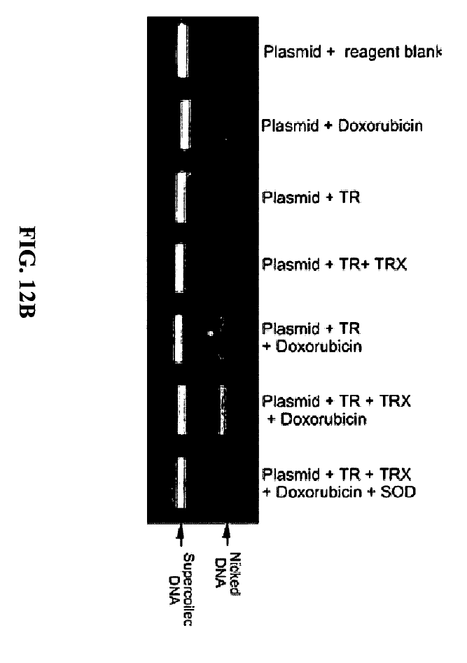

Superoxide generated by thioredoxin system with anthracyclines induce DNA damage in vitro. To further determine whether enhanced level of O₂— generated by anthracyclines in the presence of thioredoxin-thioredoxin reductase system can cause damage to plasmid DNA, closed circular plasmid DNA (pGL3) was incubated with thioredoxin system in the presence or absence of anthracyclines. As shown in FIGS. 12A-B, anthracyclines increased the level of nicks in the supercoiled DNA in the presence of thioredoxin system causing retardation in electrophoresis (FIGS. 12A-B). However in the presence of SOD the extent of DNA nicking was reduced (FIGS. 12A-B), demonstrating that O₂— does mediate DNA damage due to redox-cycling of anthracyclines by the TR-Trx system. This result also indicates that the addition of Trx offers no protection from O₂— mediated damage to the plasmid DNA.

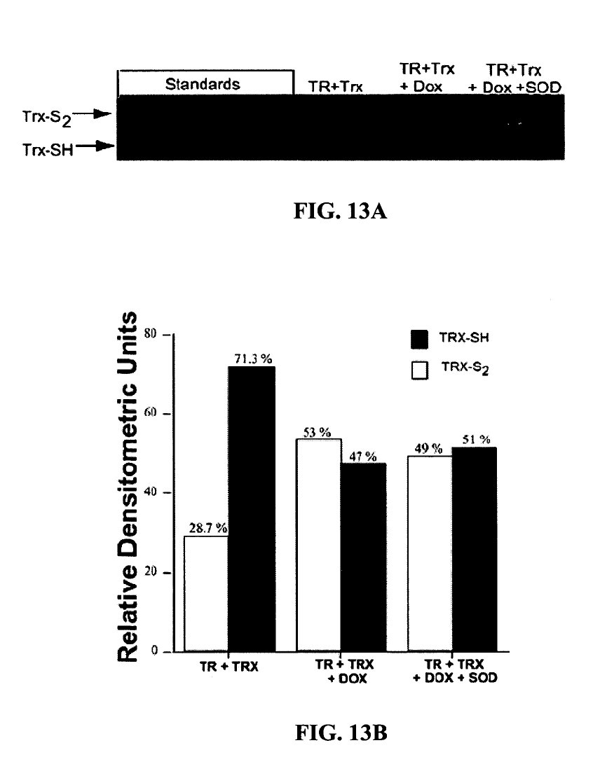

Oxidation of thioredoxin during redox-cycling of anthracylines is not inhibited by SOD. To evaluate the role of thioredoxin in redox-cycling of anthracyclines, the redox state of thioredoxin after the reaction with anthracyclines was determined. If reduced thioredoxin donates electrons for redox-cycling then more oxidized thioredoxin is expected at the end of the reaction. Thus, as demonstrated in (FIGS. 13A and 13B; lane 4), the level of Trx-S₂ was increased in the presence of doxorubicin (28.7% in untreated cells to 53% in doxorubicin treated cells). Additionally, the level of reduced thioredoxin decreased to 47% from that of 71.3% in thioredoxin-thioredoxin reductase system only. However, SOD did not prevent oxidation of thioredoxin in the presence of doxorubicin (FIG. 13B, lane 3). This suggests that O₂— does not directly oxidize thioredoxin, but oxidized thioredoxin accumulates as a result of contribution of electrons for redox-cycling.

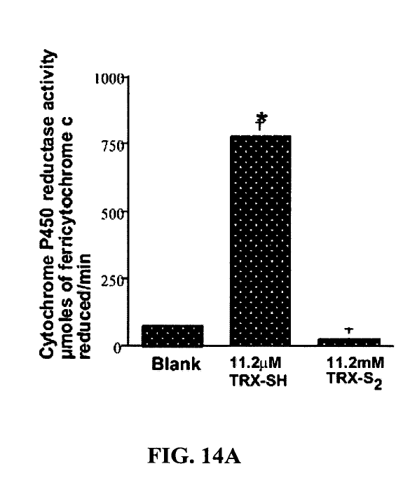

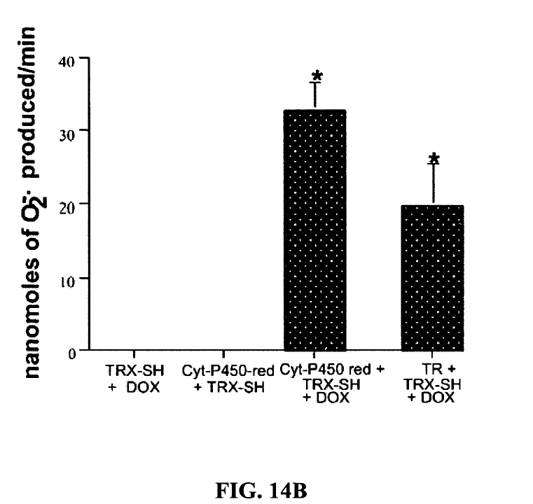

Reduced thioredoxin donates electron to cytochrome P450 reductase and enhances redox cycling of doxorubicin. Cytochrome P450 reductase is the principal enzyme that redox-cycles anthracyclines. NADPH is the source of reducing equivalents for this reaction (Das and Misra, 1992). The observation that thioredoxin system can enhance the redox-cycling of anthracyclines prompted the examination of whether reduced thioredoxin alone can be an electron donor for cytochrome P450 reductase for redox-cycling of anthracyclines. The experiments showed that addition of 11.2 μM Trx-SH can induce cytochrome P450 reductase activity in the absence of NADPH, while addition of 11.2 μM Trx-S₂ did not show any detectable cytochrome P450 reductase activity (FIG. 14A). It was further analyzed whether this activation of cytochrome P450 reductase is sufficient to redox-cycle doxorubicin by measuring the SOD inhibitable rate of O₂— generation. As presented in FIG. 14B cytochrome P450 reductase generated O₂— in the presence of 10 μM doxorubicin using 11.2 RM Trx-SH alone as electron donor in the absence of NADPH. In similar reactions (FIG. 14B, lane 4) the inventors showed that thioredoxin reductase can also redox-cycle doxorubicin using 11.2 μM Trx-SH as an electron donor. Reduced thioredoxin is more efficient with cytochrome P450 reductase in redox cycling of doxorubicin compared to E. coli thioredoxin reductase, since the final concentration of both thioredoxin reductase and cytochrome P450 reductase was 10 nM in these assays.

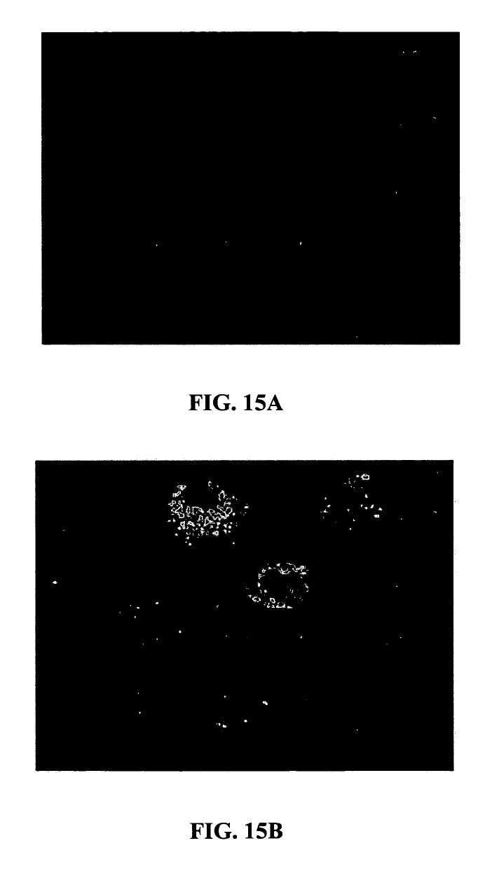

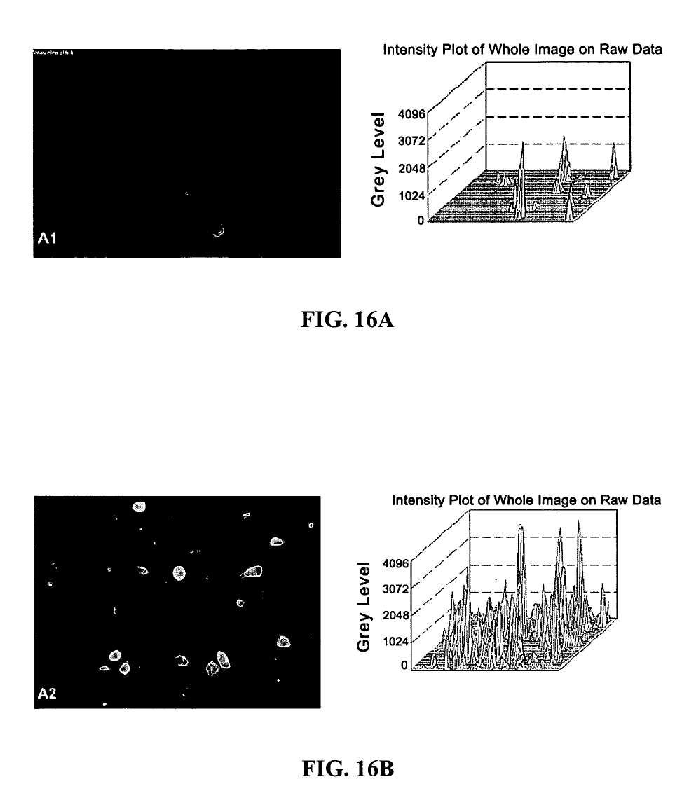

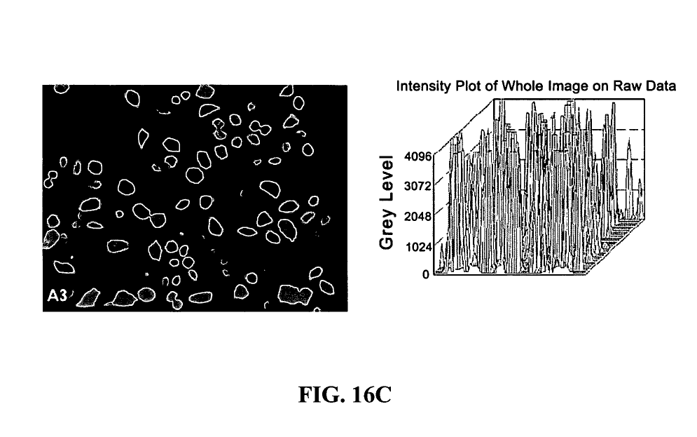

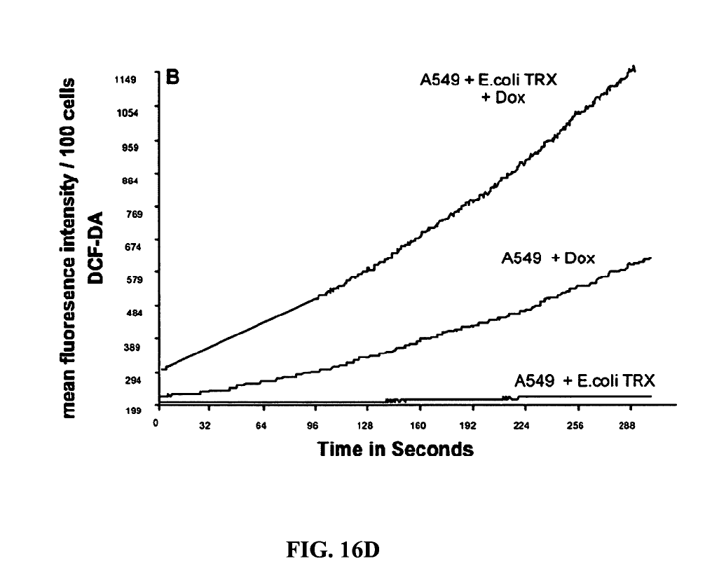

Externally added E. coli thioredoxin increases ROS generation in response to daunomycin in lung adenocarcinoma A549 cells. If E. coli thioredoxin enhances O₂— generation due to redox-cycling of daunomycin, it is expected that externally added thioredoxin (if it can enter cells) would also increase ROS generation in response to anthracyclines in vivo. It has been previously demonstrated that E. coli thioredoxin can enter A549 lung adenocarcinoma cells (Das et al., 1997). Therefore, A549 cells were used for this study. E. coli thioredoxin can act as a substrate for human thioredoxin reductase, although its K. is quite high (35 μM). Therefore, when a higher concentration of E. coli thioredoxin enters the cell it can be reduced by human thioredoxin reductase. It was previously shown that E. coli thioredoxin can remain in a reduced state in A549 cells (Das et al., 1997). Thus, it was determined whether externally added E. coli thioredoxin enhanced ROS generation in response to daunomycin. A549 cells were incubated with E. coli thioredoxin for 16 hr, and the uptake of E. coli thioredoxin by A549 cells determined using confocal microscopy (FIGS. 15A-15B) as described in the experimental procedures above. Confocal microscopy with an anti-E. coli thioredoxin antibody (FIG. 15B) demonstrated that E. coli thioredoxin entered the A549 cells and was mostly localized in the cytoplasm. Next, E. coli thioredoxin enriched A549 cells were treated with or without 1 μM concentration of daunomycin. No ROS were detected in A549 cells treated with only E. coli thioredoxin (FIG. 16A). When A549 cells were treated with daunomycin, ROS generation was increased (FIG. 16B). However, when cells were pretreated with E. coli thioredoxin followed by treatment with a 1 μM concentration of daunomycin, a significantly higher level of ROS generation was observed (FIG. 16C). The intensity of DCF-DA fluorescence was measured at specific times up to 300 s after the addition of drug in time-matched experiments, and the data plot (FIG. 16D) indicates higher mean fluorescence intensity of DCF-DA in A549 cells pretreated with E. coli Trx over the period of time compared to cells treated with doxorubicin alone. The results suggest that E. coli thioredoxin can enhance redox-cycling of anthracyclines in human cells.

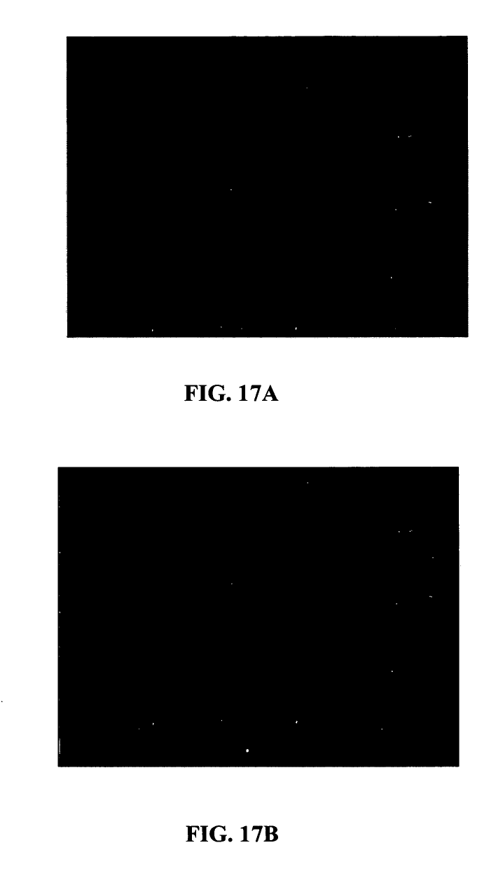

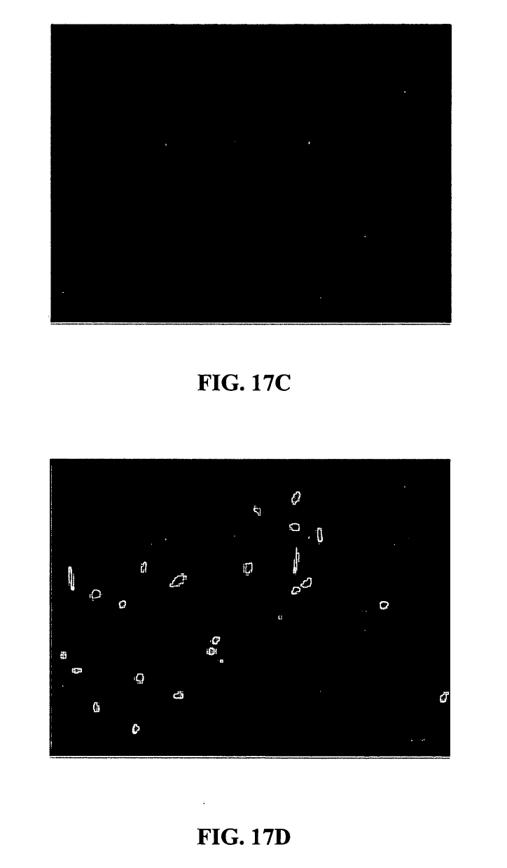

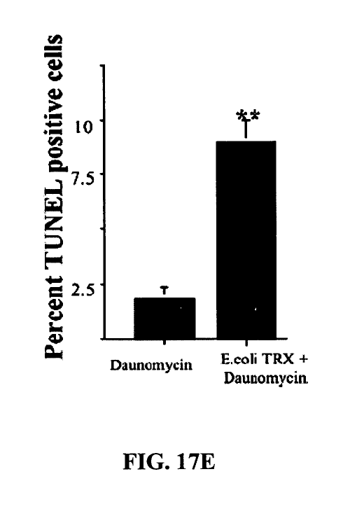

Increased TUNEL positive nuclei in A549 cells treated with E. coli thioredoxin in response to daunomycin. To determine whether anthracyclines in the presence of E. coli thioredoxin also induce DNA strand breaks (nicks) in vivo, A549 cells were incubated with E. coli thioredoxin followed by detection of DNA nicks using TUNEL (TdT-mediated dUTP-X nicks end labeling) assay as described above. As shown in FIG. 17, A549 cells treated with E. coli thioredoxin showed an increase in TUNEL positive nuclei in response to daunomycin. This experiment suggests that externally added E. coli thioredoxin can also enhance redox-cycling of anthracyclines resulting in apoptotic DNA strand breaks in vivo.

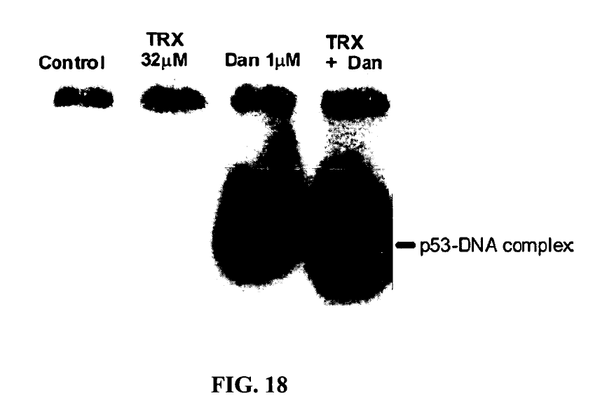

Enhanced p53 DNA binding in response to daunomycin in A549 cells enriched with E. coli Trx. p53 is a sequence-specific transcription factor that is known to be activated in response to DNA damage, and DNA damage induced by anthracyclines is known to activate p53 DNA binding [16]. Therefore p53 DNA binding could be used as a marker for the extent of DNA damage induced by anthracyclines. As demonstrated in FIG. 18 increased p53 DNA binding was observed in daunomycin-treated A549 cells enriched with E. coli Trx. This finding shows that at low physiological doses of anthracyclines, E. coli Trx can increase DNA damage in vivo.

### Example 5

### Discussion

In the present invention it has been demonstrated that increased expression of thioredoxin increased apoptosis in MCF-7 cells in response to anthracycline i.e., daunomycin. The expression and DNA binding of p53 protein was also increased in response to daunomycin in Trx9 cells compared to vector control cells. Additionally, there was increased production of O₂— in extracts of Trx9 cells in response to daunomycin. DPIC, a general reductase inhibitor decreased the generation of O₂— as well as p53-DNA binding in response to daunomycin in Trx9 cells. NADPH-dependent reductase inhibitors inhibited O₂— generation in Trx9 cells suggesting that thioredoxin provide reducing equivalents to reductases. This was verified by the fact that dominant-negative expression of mutant thioredoxin lacking its redox-active function did not increase p53 protein in response to daunomycin (FIG. 8A). These results demonstrated that thioredoxin can increase the redox-cycling and enhance the apoptotic potential of anthracyclines.

The data also suggested that increased expression of cytochrome p450 reductase may enhance the redox-cycling of anthracyclines, whereas decreased redox-cycling in cells expressing mutant thioredoxin lacking redox-active cysteines result in decreased p53 expression. Daunomycin-induced p53 DNA binding or p53 (ser-15) phosphorylation was decreased in response to MnTBAP treatment which suggested that removal of superoxide radical is not sufficient to decrease daunomycin mediated redox-cycling. On the other hand, treatment of cells with the general reductase inhibitor DPIC completely abolished p53 DNA binding and p53 (ser-15) phosphorylation suggesting that redox-cycling of daunomycin in Trx9 cells depends on the activity of bioreductive enzymes. Additionally, the data in FIG. 5A indicates that O₂— generation in Trx9 cells depends on the presence of thioredoxin. Thus, it was demonstrated that thioredoxin provides reducing equivalents to a bioreductive enzyme that enhances the redox-cycling of anthracyclines.

It was also determined whether cytochrome P450 reductase can use reduced thioredoxin as an alternative source for electron using A549 cells. The results (FIG. 14A) showed that cytochrome P450 reductase is activated by reduced thioredoxin in the absence of NADPH, while oxidized thioredoxin is not. It was further investigated whether reduced thioredoxin would also enhance the redox-cycling of anthracyclines by cytochrome P450 reductase in A549 cells. The results (FIG. 14B) demonstrated that reduced thioredoxin can effectively enhance the redox-cycling of anthracyclines by cytochrome P450 reductase as well. The efficiency of enhancing the redox cycling by cytochrome P450 reductase with respect to doxorubin was higher when compared to equimolar concentrations of E. coli thioredoxin reductase using reduced thioredoxin as the source of electron.

Thus, the present invention demonstrates that thioredoxin is involved in the redox-cycling of anthracyclines by providing electrons to bioreductive enzymes, which results in increased semiquinone radical production, and the activation of p53 mediated apoptosis of cancer cells. Thioredoxin is a redox protein capable of undergoing both oxidation and reduction reactions, which allows it to function as pro-oxidant in the redox-cycling of anthracycline antibiotics. The present invention also shows that the redox cycling reaction catalyzed by thioredoxin reductase can be enhanced by thioredoxin.

### References

The following references, to the extent that they provide exemplary procedural or other details supplementary to those set forth herein, are specifically incorporated herein by reference:
U.S. Pat. Application 20010053845
U.S. Pat. Application 20020137694
U.S. Pat. Application 20030023052
U.S. Pat. Application 20030125268
U.S. Pat. No. 3,988,315
U.S. Pat. No. 4,039,736
U.S. Pat. No. 4,127,714
U.S. Pat. No. 4,144,329
U.S. Pat. No. 4,169,142
U.S. Pat. No. 4,316,011
U.S. Pat. No. 4,684,611
U.S. Pat. No. 4,737,583
U.S. Pat. No. 4,952,500
U.S. Pat. No. 5,302,523
U.S. Pat. No. 5,322,783
U.S. Pat. No. 5,384,253
U.S. Pat. No. 5,399,363
U.S. Pat. No. 5,464,765
U.S. Pat. No. 5,466,468
U.S. Pat. No. 5,538,877
U.S. Pat. No. 5,538,880
U.S. Pat. No. 5,543,158
U.S. Pat. No. 5,550,318
U.S. Pat. No. 5,563,055
U.S. Pat. No. 5,580,859
U.S. Pat. No. 5,589,466
U.S. Pat. No. 5,610,042
U.S. Pat. No. 5,641,515
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,846,225
U.S. Pat. No. 5,846,233
U.S. Pat. No. 5,945,100
U.S. Pat. No. 5,981,274
U.S. Pat. No. 5,994,624
Asher et al., Proc. Natl. Acad. Sci. USA, 98:1188-1193, 2001.
Azzi et al., Biochem. Biophys. Res. Commun., 65:597-603, 1975.
Baichwal and Sugden, In: Gene Transfer, Kucherlapati (Ed.), New York, Plenum Press, 117-148, 1986.
Berggren et al., Anticancer Res., 16:3459-3466, 1996.
Berggren et al., Arch. Biochem. Biophys., 392:103-109, 2001.
Berglund and Holmgren, J. Biol. Chem., 250:2778-2782, 1975.
Bironaite et al., Biochim. Biophys. Acta, 1383:82-92, 1998.
Bjomstedt et al., J. Biol. Chem., 269:29382-29384, 1994.
Bolton et al., Chem. Res. Toxicol., 13:135-160, 2000.
Bruns and Karplus, J. Mol. Biol., 247:125-145, 1995.
Chen and Okayama, Mol. Cell Biol., 7(8):2745-2752, 1987.
Childs et al., Cancer Res., 62:4592-4598, 2002.
Cho, Hokkaido Igaku Zasshi, 74:239-248, 1999.
Das and Das, Biochem. Biophys. Res. Commun., 277:443-447, 2000.
Das and Misra, J. Biol. Chem., 267:19172-19178, 1992.
Das and White, J. Immunol. Methods, 211:9-20, 1998.
Das et al., Am. J. Respir. Cell Mol. Biol., 17:713-726, 1997.
Das, J. Biol. Chem., 276:4662-4670, 2001.
Fechheimer et al., Proc. Natl. Acad. Sci. USA, 84:8463-8467, 1987.
Fernando et al., Eur. J. Biochem., 209:917-922, 1992.
Fraley et al., Proc. Natl. Acad. Sci. USA, 76:3348-3352, 1979.
Fujii et al., Cancer , 68:1583-1591, 1991.
Gallegos et al., Cancer Res., 56:5765-5770, 1996.
Gardner et al., Arch. Biochem. Biophys., 325:20-28, 1996.
Gasdaska et al., Cell Growth Differ., 6:1643-1650, 1995.
Gluck, Cancer Control, 9:16-27, 2002.
Gopal, Mol. Cell. Biol., 5:1188-1190, 1985.
Graham and Van Der Eb, Virology, 52:456-467, 1973.
Gutierrez, Arch. Biochem. Biophys., 223:68-75, 1983.
Gutierrez, Front Biosci., 5:D629-638, 2000.
Harland and Weintraub, J. Cell Biol., 101:1094-1099, 1985.
Holmgren and Bjornstedt, Methods Enzymol., 252:199-208, 1995.
Holmgren, Annu. Rev. Biochem., 54:237-271, 1985.
Holmgren, J. Biol. Chem., 264:13963-13966, 1989.
Holmgren, Methods Enzymol., 107:295-300, 1984.
Holmgren, Structure, 3:239-243, 1995.
Holmgren and Lyckeborg, Proc. Natl. Acad. Sci. USA, 7:5149-5152, 1980.
Hrdina et al., Acta Medica, 43:75-82, 2000.
Husbeck and Powis, Carcinogenesis. 23:1625-1630, 2002.
Hwangetal., Nat. Med., 7:1111-1117, 2001.
Iyanagi and Yamazaki, Biochim. Biophys. Acta, 172:370-381, 1969.
lyanagi and Yamazaki, Biochim. Biophys. Acta, 216:282-294, 1970.
Kaeppler et al., Plant Cell Reports, 9:415-418, 1990.
Kaneda et al., Science, 243:375-378, 1989.
Kato et al, J. Biol. Chem., 266:3361-3364, 1991.
Keizer et al., Pharmacol. Ther., 47:219-231, 1990.
Komiyama et al., Biochem. Pharmacol., 34:977-983 29, 1985.
Lundstrom and Holmgren, J. Biol. Chem., 265:9114-9120, 1990.
Makino et al., J. Clin. Invest., 98:2469-2477, 1996.
Mathews et al., J. Pharmacol. Exp. Ther., 235:186-190, 1985.
Mau and Powis, Biochem. Pharmacol., 43:1613-1620, 1992.
Mau and Powis, Free Radic. Res. Commun., 8:365-372, 1990.
Maya et al., Genes Dev., 15:1067-1077, 2001.
Meyer et al., EMJO. J. , 12:2005-2015, 1993.
Mitsui etal., Biochem. Biophys. Res. Commun., 186:1220-1226, 1992.
Mordente et al., IUBMB Life, 52:83-88, 2001.
Nakamura et al., Cancer, 69:2029-2079, 1992.
Nakamura et al., Immunol. Lett., 42:75-80, 1994.
Newman et al., J. Exp. Med., 180:359-363, 1994.
Ngo et al., Chem. Res. Toxicol., 11:360-368, 1998.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods EnzymoL, 149:157-176, 1987.
Nordberg et al., J. Biol. Chem., 273:10835-10842, 1998.
Oblong etal., J. Biol. Chem., 269:11714-11720, 1994.
Oblong etal., J. Biol. Chem., 269:11714-11720, 1994.
Omirulleh et al., Plant Mol. Biol., 21(3):415-28, 1993.
Osada et al., J. Biol. Chem., 277:23367-23373, 2002.
Pan and Bachur, Mol. Pharmacol., 17:95-99, 1980.
Pan et al., Mol. Pharmacol., 19:184-186, 1981.
PCT Application WO 94/09699
PCT Application WO 9506128
Potrykus et al., Mol. Gen. Genet., 199:183-188, 1985.
Potter et al., Proc. Natl. Acad. Sci. USA, 81:7161-7165, 1984.
Powis and Montfort, Annu. Rev. Biophys. Biomol. Struct., 30:421-455, 2001
Powis et al., Chem. Biol. Interact., 24:111-112:23-34, 1998.
Remington's Pharmaceutical Sciences, 15^{th} ed., pages 1035-1038 and 1570-1580, Mack
Publishing Company, Easton, Pa., 1980.
Ridgeway, In: Vectors: A survey of molecular cloning vectors and their uses, Rodriguez and Denhardt (Eds.), Stoneham: Butterworth, 467-492, 1988.
Rippe et al., Mol. Cell Biol., 10:689-695, 1990.
Sadji-Ouatas et al., Biochem. J, 364:881-885, 2002.
Sasada et al., J. Clin. Invest., 97:2268- 2276, 1996.
Schallreuter and Wood, Biochem. Biophys Res. Commun., 136:630-637, 1986.
Schenk et al., Proc. Natl. Acad. Sci. USA, 91:1672-1676, 1994.
Spector et al., J. Biol. Chem., 263:4984-4990, 1988.
Tagaya et al., EMJO. J. , 8:757-764, 1989.
Temin, In: Gene Transfer, Kucherlapati (Ed.), NY, Plenum Press, 149-188, 1986.
Tur-Kaspa et al., Mol. Cell Biol., 6:716-718, 1986.
Wakasugi et al., Proc. Natl. Acad. Sci. USA, 87:8282-8286, 1990.
Weiss, Semin. Oncol., 19:670-686, 1992.
Wong et al., Gene, 10:87-94, 1980.
Wu and Wu, Biochemistry, 27:887-892, 1988.
Wu and Wu, J. Biol. Chem., 262:4429-4432, 1987.
Yang et al., Science, 275:1129-1132, 1997.
Yodoi and Tursz, Adv. Cancer Res., 57:381-411, 1991.
Yokomizo et al., Cancer Res., 55:4293-4296, 1995.

1. A method of treating a subject comprising administering to the subject a therapeutically effective amount of a thioredoxin or a thioredoxin-like molecule and providing to the subject an anticancer therapy.

2. The method of claim 1, wherein the thioredoxin or thioredoxin-like molecule enhances the redox-cycling of the anticancer therapy thereby promoting cell killing.

3. The method of claim 1, wherein the subject has cancer.

4. The method of claim 3, wherein the cancer is a premalignant cancer.

5. The method of claim 3, wherein the cancer is a malignant cancer.

6. The method of claim 3, wherein the cancer is a metastatic cancer.

7. The method of claim 3, wherein the cancer is a breast cancer, lung cancer, head and neck cancer, bladder cancer, bone cancer, bone marrow cancer, brain cancer, colon cancer, esophageal cancer, gastrointestinal cancer, gum cancer, kidney cancer, liver cancer, nasopharynx cancer, ovarian cancer, prostate cancer, skin cancer, stomach cancer, testis cancer, tongue cancer; or uterine cancer.

8. The method of claim 7, wherein the cancer is breast cancer.

9. The method of claim 1, wherein the anticancer therapy comprises administration of a chemotherapeutic agent.

10. The method of claim 9, wherein the thioredoxin or thioredoxin-like molecule enhances the redox-cycling of the chemotherapeutic agent.

11. The method of claim 9, wherein the chemotherapeutic agent is an anthracycline.

12. The method of claim 11, wherein the anthracycline is doxorubicin.

13. The method of claim 11, wherein the anthracycline is daunomycin.

14. The method of claim 1, wherein the anticancer therapy comprises administration of a radiotherapeutic agent.

15. The method of claim 14, wherein the anticancer therapy comprises radiation.

16. The method of claim 1, wherein thioredoxin or thioredoxin-like molecule is administered before the anticancer therapy.

17. The method of claim 1, wherein the thioredoxin or thioredoxin-like molecule is administered at the same time as the anticancer therapy.

18. The method of claim 1, wherein the thioredoxin or thioredoxin-like molecule is administered once or more than once.

19. The method of claim 1, wherein the thioredoxin or thioredoxin-like molecule is administered intravenously, intradermally, intramuscularly, intraarterially, intralesionally, percutaneously, subcutaneously, or by an aerosol.

20. The method of claim 1, wherein the thioredoxin or thioredoxin-like molecule is administered directly to the tumor.

21. The method of claim 1, wherein the anticancer therapy is provided once or more than once.

22. The method of claim 1, wherein the anticancer therapy comprises administration of an anticancer agent and the anticancer agent is administered intravenously, intradermally, intramuscularly, intraarterially, intralesionally, percutaneously, subcutaneously, or by an aerosol.

23. The method of claim 1, further comprising delivering to a subject an expression construct comprising a nucleic acid encoding a thioredoxin or a thioredoxin-like molecule.

24. The method of claim 23, wherein the expression construct is a viral vector.

25. The method of claim 24, wherein the viral vector is an adenoviral vector, an adeno-associated viral vector, a retroviral vector, a lentiviral vector, a herpes viral vector, polyoma viral vector or hepatitis B viral vector.

26. The method of claim 24, wherein the viral vector is an adenoviral vector.

27. The method of claim 1, further comprising a gene therapy.

28. The method of claim 27, further comprising an adenoviral gene therapy.

29. The method of claim 1, wherein the subject is a mammal.

30. The method of claim 29, wherein the mammal is a human.

31. A method of sensitizing a cancer cell to an anticancer agent comprising providing an effective amount of a thioredoxin or a thioredoxin-like molecule and an anticancer therapy to the cancer cell.

32. The method of claim 31, wherein the thioredoxin or thioredoxin-like molecule enhances the redox-cycling of the anticancer agent.

33. The method of claim 31, further comprising inducing apoptosis in the cancer cell.

34. The method of claim 31, wherein the cancer cell is a premalignant cancer cell.

35. The method of claim 31, wherein the cancer cell is a malignant cancer cell.

36. The method of claim 31, wherein the cancer cell is a metastatic cancer cell.

37. The method of claim 31, wherein the cancer cell is a breast cancer cell, lung cancer cell, head and neck cancer cell, bladder cancer cell, bone cancer cell, bone marrow cancer cell, brain cancer cell, colon cancer cell, esophageal cancer cell, gastrointestinal cancer cell, gum cancer cell, kidney cancer cell, liver cancer cell, nasopharynx cancer cell, ovarian cancer cell, prostate cancer cell, skin cancer cell, stomach cancer cell, testis cancer cell, tongue cancer cell, or uterine cancer cell.

38. The method of claim 37, wherein the cancer cell is breast cancer cell.

39. The method of claim 31, wherein the anticancer therapy comprises administration of a chemotherapeutic agent.

40. The method of claim 39, wherein the chemotherapeutic agent is an anthracycline.

41. The method of claim 40, wherein the anthracycline is doxorubicin.

42. The method of claim 40, wherein the anthracycline is daunomycin.

43. The method of claim 31, wherein the anticancer therapy comprises administration of a radiotherapeutic agent.

44. The method of claim 43, wherein the anticancer therapy comprises radiation.

45. The method of claim 31, wherein the cell is located in a tissue culture.

46. The method of claim 31, wherein the cell is located in a subject.

47. A method of ameliorating negative side-effects of an anticancer therapy in a subject comprising administering to the subject a therapeutically effective amount of a thioredoxin or a thioredoxin-like molecule and an anticancer therapy.

48. A pharmaceutical combination comprising a thioredoxin or a thioredoxin-like molecule and an anticancer agent.

49. The pharmaceutical combination of claim 48, wherein the anticancer agent is a chemotherapeutic agent.

50. The pharmaceutical combination of claim 49, wherein the chemotherapeutic agent is an anthracycline.

51. The pharmaceutical combination of claim 50, wherein the anthracycline is doxorubicin.

52. The pharmaceutical combination of claim 50, wherein the anthracycline is daunomycin.

53. The pharmaceutical combination of claim 48, wherein the thioredoxin or a thioredoxin-like molecule and the anticancer agent are comprised together in a pharmaceutically acceptable excipient.

54. The pharmaceutical combination of claim 48, wherein the thioredoxin or a thioredoxin-like molecule and the anticancer agent are each separately comprised in a pharmaceutically acceptable excipient.
